(19) United States
(12) Patent Application Publication
(10) Pub. No.: US 2017/0209544 A1
(43) Pub. Date: Jul. 27, 2017

(54) A PEPTIDE THERAPY TO COUNTERACT INSULIN RESISTANCE AND TYPE 2 DIABETES

(71) Applicants: The Regents of the University of California, Oakland, CA (US); Indian Institute of Technology Madras, Chennai (IN)

(72) Inventors: Sushil K. Mahata, San Diego, CA (US); Gautam K. Bandyopadhyay, San Diego, CA (US); Sanjib Senapati, Chennai (IN)

(21) Appl. No.: 15/326,348

(22) PCT Filed: Jul. 15, 2015
(86) PCT No.: PCT/US15/40533

§ 371 Date: Jan. 13, 2017

Related U.S. Application Data
(60) Provisional application No. 62/024,860, filed on Jul. 15, 2014.

(51) Int. Cl. A61K 38/22; A61K 9/00; A61K 45/06

(52) U.S. Cl. CPC A61K 38/22; A61K 45/06; A61K 9/0019

(57) ABSTRACT

Compositions comprising an antagonist of pancreastatin are provided. The beneficial use of the compositions for treating insulin resistance, diabetes, especially type II diabetes, inflammation, obesity, non-alcoholic fatty liver disease, atherosclerosis and cardiovascular diseases is described as well.

### CROSS-REFERENCE TO RELATED APPLICATIONS

This application claims a benefit of priority to the U.S. Provisional Application 62/024,860, filed Jul. 15, 2014, the entire disclosure of which is incorporated herein.

### FIELD OF THE INVENTION

This invention relates to compositions and methods for treatment of type 2 diabetes as well as to compositions and methods for treatment of insulin resistance.

### BACKGROUND

Insulin is a hormone produced by the pancreas. It regulates uptake of glucose by cells in humans and other mammals. In some people, cells fail to respond to insulin. This condition is called insulin resistance (IR).

The insulin resistance may lead to high blood glucose levels, pre-diabetes, type 2 diabetes, other types of diabetes, high blood pressure, cardiovascular diseases, obesity, inflammation, depression and many other diseases. It will be understood that the term “insulin resistance” includes a variety of physiological conditions which manifest itself by at least one of the following symptoms: the increased level of insulin in a patient's blood over an average level typical for humans and/or at least partial failure of a patient's body to properly metabolize carbohydrates in response to stimulation with insulin.

Various patients, including those suffering from type 2 diabetes, obesity and age-related diseases may benefit from a medication which ameliorates the effects of insulin resistance.

The Chromogranin A (human CHGA/mouse Chga) pro-protein undergoes proteolysis and gives rise to bioactive peptides including the antihypertensive catestatin (CHGA₃₅₂₋₃₇₂) and the diabetogenic pancreastatin (PST: CHGA₂₅₀₋₃₀₁). The Chga deficient mice (Chga-KO) are obese, hyperadrenergic and hypertensive. They display elevated levels of circulating leptin and catecholamines but lower levels of IL-6 and Mcp-1. Despite these abnormalities, Chga-KO mice exhibit enhanced insulin sensitivity, a phenotype masked by supplementing PST.

PST regulates hepatic insulin signaling through cPKC and Srebp-1c. Increased plasma PST levels in diabetic populations correlate with insulin resistance. Similarly, increased circulating levels of PST in diet-induced obese (DIO) and diabetic db/db mice are associated with insulin resistance. Despite high levels of plasma leptin and catecholamines, Chga-KO mice are obese owing to peripheral leptin and catecholamine resistance.

Since normal chow diet (NCD)-fed Chga-KO mice displayed increased insulin sensitivity, Chga-KO mice may be able to maintain insulin sensitivity when exposed to the dysglycemic stress of a high fat diet (HFD). The hallmarks of insulin resistance in DIO mice are obesity, hyperinsulinemia and increased inflammation. Suppression of inflammation in DIO mice can improve insulin sensitivity. For example, rosiglitazone can decrease inflammation and increase insulin sensitivity in DIO mice without reducing obesity significantly. Chga-KO mice are obese and presumably would become more obese after HFD feeding.

While some progress has been made with stabilizing and treating insulin resistant and type 2 diabetic patients, the main two drug groups are metformin and thiazolidinediones (TZDs). However, these drugs are not without some side effects. For example, it has been reported recently that pioglitazone, one of the TZDs, increases the incidence of bladder cancer.

Another option for controlling blood glucose levels is insulin. However, it is unlikely that insulin can be beneficial to patients with insulin resistance and diabetic patients whose diabetes is not caused by the insulin shortage. There are other peptides such as adiponectin which are being tested for anti-diabetic effects. However, insulin and adiponectin are endogenous hormones and persistent activation of their receptors by therapeutic levels of hormones may cause receptor desensitization, insulin resistance and adiponectin resistance, which may lead to unexpected side effects.

Thus, there remains the need for new compositions and methods for treating type 2 diabetes and insulin resistance.

### INVENTION SUMMARY

One embodiments provides a composition comprising a peptide comprising the amino acid sequence KGEQEHSQQKEEEEEMAVVPQGLFRG-AMIDE (SEQ ID NO. 1) and at least one excipient.

Another embodiment provides a composition comprising a peptide which consists of the amino acid sequence KGEQEHSQQKEEEEEMAVVPQGLFRG-AMIDE (SEQ ID NO. 1).

Further embodiment provides a composition in which a peptide comprises the amino acid sequence in which at least one of the amino acids from KGEQEHSQQKEEEEEMAVVPQGLFRG-AMIDE (SEQ ID NO. 1) is substituted or deleted. At least some of the embodiments provide a composition in which a peptide comprises the amino acid sequence in which at least one of the amino acids from KGEQEHSQQKEEEEEMAVVPQGLFRG-AMIDE (SEQ ID NO. 1) is substituted or deleted and the peptide retains at least 50% binding affinity to the insulin receptor of the binding activity for the peptide with SEQ ID NO. 1 to the insulin receptor.

Any of these compositions may further comprise additionally any of the following drugs: an anti-inflammatory drug, metformin, thiazolidinedione or insulin.

Any of the these compositions can be formulated for oral administration, or intravenous or intramuscular injection into a patient.

These compositions can be used for treating a patient from any of the following diseases: insulin resistance, diabetes, inflammation, obesity, non-alcoholic fatty liver disease, atherosclerosis and other cardiovascular diseases.

Various patients can benefit from treatment with the compositions. At least some of these patients are selected from the group of patients whose fasting insulin level is 9.0 mlU/ml or higher.

At least in some of the treatment protocols, a patient can be treated with any of the above described compositions at the amount ranging from 5 mg/day to 1 g/day.

### BRIEF DESCRIPTION OF THE DRAWINGS

FIGS. 1A-1B show that plasma PST level is increased in (FIG. 1A) human subjects with T2DM (FIG. 1A) and in mice with DIO (60% fat for 16 weeks) and db/db mice (FIG. 1B).

FIGS. 2A-2B show that PSTv1 (the peptide with SEQ ID NO. 1) regulate glucose tolerance (FIG. 2A) and sensitivity to insulin (FIG. 2B) in DIO mice.

FIGS. 3A-3B show GIR (FIG. 3A) and suppression (%) of HGP in HFD-fed wild-type & Chga-KO mice (FIG. 3B).

FIGS. 4A-4D show the effects of PSTv1 (the peptide with SEQ ID NO. 1) on (FIG. 4A) plasma and (FIG. 4B) liver NO and (FIG. 4C) liver glycogen content in DIO mice, and (FIG. 4D) PST-induced NO production in hepatocytes.

FIGS. 5A-5I show the effects of PSTv1 (the peptide with SEQ ID NO. 1) on IL-1beta mRNA in liver (FIG. 5A), MCP1 mRNA in liver (FIG. 5B), IL-6 mRNA in liver (FIG. 5C), IL-1 beta protein in serum (FIG. 5D), MCP-1 protein in serum (FIG. 5E), G-CSF protein in serum (FIG. 5F), TNF-Alpha mRNA in the adipose tissue (FIG. 5G), MCP-1 mRNA in the adipose tissue (FIG. 5H) and IL-6 mRNA in the adipose tissue (FIG. 5I).

FIGS. 6A-6D show the effects of PSTv1 (the peptide with SEQ ID NO. 1) on AKT expression (FIG. 6A), AKT phosphorylation (FIG. 6B), JNK expression (FIG. 6C) and phosphorylation of eNOS in liver (FIG. 6D).

FIG. 7A shows the effects of various PST variant peptides on PST-induced glucose uptake in adipocytes. FIG. 7B is a schematic diagram showing GRP78 bound with UPR in NCD mice and FIG. 7C shows dissociation of GRP78 and UPR for activation of UPR signaling in HFD mice. FIG. 7D is a micrograph of the ER ultrastructure of liver in mice kept on normal chow (NCD). FIG. 7E is a micrograph of the ER ultrastructure of liver in mice in which obesity was induced with a high fat diet (DIO). FIG. 7F is a micrograph of the ER ultrastructure of liver in DIO mice treated with PSTv1. FIG. 7G reports the ER lumen width for all three groups of mice (n=24). FIGS. 7H, 7I and 7J are immunoblots showing changes in the expression of PERK, elf2a and IRE1α, respectively. *: π<0.05; **: π<0.01; ***: p<0.001 #: not significant.

FIGS. 8A and 8B show chronic PSTv1 (the peptide with SEQ ID NO. 1) blocks the ability of PST to suppress the glucose tolerance of KO-NCD mice. GTT is shown in FIG. 8A and the corresponding AUC is shown in FIG. 8B. FIGS. 8C, 8D and 8E are micrographs showing the ultrastructure of liver in NCD (FIG. 8C), DIO (FIG. 8D), and DIO+CST (FIG. 8E) mice showing juxtaposition of ER and mitochondria by arrows. Morphometric analyses of the distance between ER and mitochondria are shown in FIG. 8F (n=24). FIG. 8G is a schematic diagram showing physical contacts between endoplasmic reticulum and mitochondria through mitochondria associated membranes (MAM). FIG. 8H are immunoblot analyses showing changes in the expression of GRP75. *: p<0.05; ***: p<0.001; #: not significant.

FIGS. 9A-9C show structural comparison between insulin (FIG. 9A), PST-WT (FIG. 9B) and PST-NΔ3, the peptide with SEQ ID NO. 1 (FIG. 9C). FIG. 9D is an immunoblot assay (n=4) showing expression of mitochondrial complex proteins in NCD, DIO and DIO+PStv1 mice. FIGS. 9E-9H report immunoblot analyses showing changes in the expression of Complex I, Complex II, Complex III, and Complex V, respectively. *: p<0.05; **: p<0.01; #: not significant.

FIGS. 10A and 10B show the molecular docking of wild type PST and PST-NΔ3 (the peptide with SEQ ID NO. 1) to insulin receptor. FIG. 10A shows the functional dimeric structure of the insulin receptor (IR) is shown in surface mode. FIG. 10B shows that out of the two equivalent binding pockets in IR, one is zoomed to depict the binding of the peptides in the insulin binding pocket. Insulin is shown as helix 1, while PST-WT and PST-NΔ3 are shown, respectively, as helix 2 and helix 3. The αCT helix of the insulin receptor is highlighted as helix 4.

FIGS. 11A-11G show that Chga-KO mice display elevated body weight gain on NCD and HFD. FIG. 11A is a graph of body weights of WT and Chga-KO mice (with mixed genetic background) on NCD from week 7 until week 22 (2-way ANOVA: Strain, p<0.0001; Age, p<0.0001; Interaction, p<0.004; n=9). FIG. 11B is a diagram of the initial weight at week 7 and final weight at week 22. FIG. 11C is a graph of body weights of WT and Chga-KO mice from week 7 until week 22. FIG. 11E is a diagram of effects of PST administration to WT and Chga-KO mice on NCD for 2 weeks (from week 19 to week 21) on body weight gain. FIG. 11F is a diagram of plasma PST levels in 6 month old WT-NCD mice, or WT-DIO or in obese-diabetic db/db mice (all mice with C57/BL6 background) (n=6). FIG. 11G is a diagram of plasma leptin levels for four month old WT and Chga-KO mice with mixed genetic background, which were fed NCD or HFD for 12 weeks.

FIGS. 12A-12L show that Chga-KO mice on HFD display improve glucose tolerance and insulin sensitivity, dependent upon the absence of PST. WT-DIO and KO-DIO male mice with mixed genetic background were fasted for 12 hrs and subjected to IP-GTT (FIG. 12A), oral GTT (FIG. 12C), or IP-ITT (FIG. 12E), and AUC for glucose excursions were determined (FIGS. 12B, 12D and 12F). In FIG. 12E, WT-DIO and KO-DIO mice were treated with intraperitoneal PST and subjected to GTT. GTT and the corresponding AUC are shown in (FIG. 12E) and (FIG. 12F), respectively. Body weight-matched WT-DIO and KO-DIO mice were fasted for 12 hrs and subjected to clamp studies to determine (FIG. 12G) glucose infusion rate (GIR), (FIG. 12H) glucose disposal rate (GDR), (FIG. 12I) insulin-stimulated GDR (IS-GDR) and (FIG. 12J) % suppression of hepatic glucose production (HGP) (n=8-9).

FIGS. 13A-13H report results of treatment with the peptide with SEQ ID NO. 1 (PSTv1). WT-DIO mice were treated with saline or PSTv1, were fasted for 12 hrs and subjected to IP-GTT (FIG. 13A) and to IP-ITT assay (FIG. 13C). AUCs for glucose excursions were determined and are shown in FIGS. 13B and 13D. NCD fed WT mice WT-NCD were treated with saline or PST, fasted for 12 hrs and subjected to IP-ITT, results of which are shown in FIG. 13E. The corresponding AUC is shown in FIG. 13F. WT-NCD mice were treated with saline or PSTv1, fasted for 12 hrs and subjected to IP-ITT, results of which are shown in FIG. 13G. The corresponding AUC is shown in 13H.

FIGS. 14A-14G report data supporting the conclusion that that PSTv1 (the peptide with SEQ ID NO. 1) blocks the ability of PST to suppress the glucose tolerance of KO-NCD mice, and PST treatment reversed reduced gluconeogenic and lipid metabolic gene expression in KO-DIO mice. FIG. 14A is an IP-GTT assay for acute effects, while FIG. 14B is a corresponding AUC. FIG. 14C is an IP-GTT assay for chronic effects, while the corresponding AUC is shown in FIG. 14D. FIG. 14E reports liver glycogen at basal and during clamp in DIO mice. FIG. 14F reports chronic effects of PSTon expression of hepatic gluconeogenic genes (Pepck and G6pase) in DIO mice. FIG. 14G reports chronic effects of PST on expression of hepatic lipid metabolic (Acc, Ppara, Cpt-1, Acox-1 and Srebp-1) genes in DIO mice.

FIGS. 15A-15F show results of experiments in which weight-matched saline-treated WT-DIO or KO-DIO and PST-treated KO-DIO mice were fasted, injected with insulin and sacrificed for tissue collection. Tissues were homogenized, lysates were subjected to SDS-PAGE and immunoblotted for p-Akt in muscle (FIG. 15A), WAT (FIG. 15B) and liver (FIG. 15C) and for p-AMPK in muscle (FIG. 15D), WAT (FIG. 15E) and liver (FIG. 15F).

FIGS. 16A-16E are immunoblots detecting phospho-p95-FoxO1 and phospho-p82-FoxO1 signals in WAT (FIG. 16A) and phospho-p82-FoxO1 signals in liver (FIG. 16B), phospho-p54-Jnk and phospho-p46-Jnk signals in WAT (FIG. 16C) and liver (FIG. 16D) and Srebp-1 in liver (FIG. 16E).

FIGS. 17A-17H show results of the experiments in which a group of KO-DIO mice were injected with saline or PST. Weight-matched saline-treated WT-DIO, KO-DIO and PST-treated KO-DIO mice were fasted for 12 hr and sacrificed. Blood was collected to measure plasma cytokine levels. Tissues were subjected to RNA extraction, cDNA preparation and RT-qPCR analysis for cytokines (FIG. 17A, n=6), pro-inflammatory genes (FIG. 17B: Cd11c & IL-12p40, n=6; FIG. 17C: iNos, n=6), and anti-inflammatory genes (FIG. 17D: Arg-1 & IL-10, n=6; FIG. 17E: Mgl-1, Mgl-2 and Ym1, n=6). The adipose tissue expression of Tnfα, IL-6 and Mcp-1 genes is shown in FIG. 17F, while plasma cytokine levels are shown in FIGS. 17G and 17H.

FIGS. 18A-18H report that PST promotes macrophage inflammation and chemotaxis. Peritoneal macrophages were isolated from WT-NCD and KO-NCD after thioglycollate injection. After 4-hr exposure to saline, LPS (100 ng/ml) or PST (100 nM), RNAs were extracted and cDNAs were prepared for qPCR analyses of Chga (FIG. 18A, n=8), iNos (FIG. 18B, n=8), Tnfα (FIG. 18C, n=8), Mcp-1 (FIG. 18D, n=8), IL-6 (FIG. 18E, n=8), IL-12p40 (FIG. 18F, n=8) and iNos genes (FIG. 18G, n=8). FIG. 18H reports the effects of PST on cell chemotaxis.

FIGS. 19A-19E report GTT and ITT experiments in mice with C57BL/6 genetic background. Three month old WT and Chga-KO mice were fed HFD for 12 weeks. Body weight-matched mice were fasted for 12 hrs and subjected to IP-GTT (FIG. 19A) and ITT (FIG. 19C) and the corresponding AUCs for glucose excursions were determined (FIGS. 19B and 19D). FIG. 19E reports results of experiments in which four month old Chga-KO mice were fed HFD for 12 months, fasted for 12 hrs, injected with PST (5 μg/g BW, IP) at −30 min and subjected to GTT after injecting glucose at 0 min. *: P<0.05, **: p<0.01, ***: p<0.001.

### DETAILED DESCRIPTION

This invention provides compositions and methods for treating at least one of the following conditions: type 2 diabetes and insulin resistance. A person of skill will appreciate that the term “insulin resistance” can be manifested to a various degree in various patients. Some manifestations of insulin resistance include: 1) a failure of patient's cells to properly metabolize carbohydrates in respond to insulin and/or 2) increased levels of insulin in patient's blood after fasting in comparison to an average fasting insulin level for the population. It will be appreciated that the average fasting insulin level in the US is believed to be at about 8.8 mlU/ml for men and at about 8.4 mlU/ml for women. Thus, fasting insulin levels above 8.8 mlU/ml may be indicative of insulin resistance.

In further embodiments, the invention provides compositions useful for treating patients suffering from at least one of the following conditions: type 2 diabetes, insulin resistance, other types of diabetes, obesity which coincides with insulin resistance, cardiovascular diseases which coincide with insulin resistance, depression, pre-diabetes and other conditions in which patient's body fails to properly metabolize carbohydrates.

One embodiment provides a composition comprising a peptide comprising the following amino acid sequence KGEQEHSQQKEEEEEMAVVPQGLFRG-amide (SEQ ID NO. 1). Other embodiment provides a composition consisting essentially of a peptide comprising the following amino acid sequence KGEQEHSQQKEEEEEMAVVPQGLFRG-amide (SEQ ID NO. 1). Other embodiment provides a composition consisting of a peptide comprising the following amino acid sequence KGEQEHSQQKEEEEEMAVVPQGLFRG-amide (SEQ ID NO. 1).

Further embodiments provide a composition comprising, consisting essentially of or consisting of a peptide consisting essentially of the following amino acid sequence KGEQEHSQQKEEEEEMAVVPQGLFRG-amide (SEQ ID NO. 1). Further embodiments provide a composition comprising, consisting essentially of or consisting of a peptide consisting of the following amino acid sequence KGEQEHSQQKEEEEEMAVVPQGLFRG-amide (SEQ ID NO. 1).

Further embodiments provide a composition comprising, consisting essentially of or consisting of a peptide consisting of an amino acid sequence with at least 99% percent homology to KGEQEHSQQKEEEEEMAVVPQGLFRG-amide (SEQ ID NO. 1).

Further embodiments provide a composition comprising, consisting essentially of or consisting of a peptide consisting of an amino acid sequence with at least 70% percent homology to KGEQEHSQQKEEEEEMAVVPQGLFRG-amide (SEQ ID NO. 1). Further embodiments provide a composition comprising, consisting essentially of or consisting of a peptide consisting of an amino acid sequence with about 70% to 100% percent homology to KGEQEHSQQKEEEEEMAVVPQGLFRG-amide (SEQ ID NO. 1).

Further embodiments provide compositions with a variant peptide in which at least one amino acid in SEQ ID NO. 1 has been substituted or deleted. In some embodiments, the variant peptide with at least one substitution or deletion has retained at least 80% binding affinity to the insulin receptor of the binding affinity of the peptide with SEQ ID NO. 1 to the insulin receptor. A person of skill will appreciate that any variant peptide with at least 50% homology to the peptide with SEQ ID NO. 1 is considered to be an equivalent of the peptide with SEQ ID NO. 1, provided that this variant peptide has retained at least 50% binding affinity to the insulin receptor of the binding affinity of the peptide with SEQ ID NO. 1 to the insulin receptor.

Further embodiments include any of the compositions comprising a peptide with SEQ ID NO. 1 and/or any variant-equivalent of the peptide with SEQ ID NO. 1, formulated with at least one excipient. Such compositions can be formulated as tablets suitable for oral treatment. In further embodiments, the compositions can be formulated as tablets with delayed release. Other suitable formulations may include formulations for intravenous injections, intramuscular injections, a liquid form for delivery with a portable pump or a liquid form for delivery with a syringe. Other formulations may include patches, creams and syrups.

In further embodiments, a pharmaceutical formulation may be prepared comprising a peptide containing SEQ ID NO. 1 and least one excipient. Yet in other embodiments, the formulation may comprise another drug or hormone commonly used for treating insulin resistance and/or diabetes in combination with a peptide containing SEQ ID NO. 1 and at least one excipient. In some embodiments, the formulation may comprise a peptide containing SEQ ID NO. 1, excipient and insulin. In other embodiments, the formulation may comprise a peptide containing SEQ ID NO. 1, excipient and metformin.

Other suitable formulations may further comprise a peptide containing SEQ ID NO. 1 and at least one drug commonly used for treating inflammation. Such anti-inflammatory drugs may include, but are not limited to, aspirin.

A peptide containing SEQ ID NO. 1 can be obtained by the solid phase synthesis. It can be further subsequently purified by reverse phase HPLC (high-performance liquid chromatography). Other variant peptides equivalent to the peptide with SEQ ID NO. 1 can be also obtained by the solid phase synthesis. These variant equivalent peptides can be also subsequently purified by reverse phase HPLC.

Other methods can also be used for making a peptide with SEQ ID NO. 1 and its equivalents. Such methods include recombinant methods in which a recombinant DNA construct expressing a peptide with SEQ ID NO. 1 or its equivalent is engineered, and is then caused to synthesize a peptide with SEQ ID NO. 1 or the equivalent. In further embodiments, a recombinant cell can be engineered which expresses a peptide with SEQ ID NO. 1. Similar recombinant methods can be utilized to express a variant peptide equivalent to the peptide with SEQ ID NO. 1.

It will be appreciated by a person of skill that suitable compositions include those in which the peptide with SEQ ID NO. 1 or its variant is further chemically modified with a tag. Such modifications may include those for increasing or decreasing the peptide's half-life, modifications for increasing or decreasing its affinity to the insulin receptor and/or to improve detection, delivery and localization in patient's body and/or inside of a cell. Suitable modifications may include replacing at least some or all L-amino acids with D-amino acids. This peptide has a longer half-life and is not easily degradable in vivo.

Other embodiments provide methods in which a peptide comprising SEQ ID NO. 1 is used for administering to a patient in need for treatment of at least one of type 2 diabetes and insulin resistance. In some of these methods, a patient is tested for at least one of the following: blood glucose levels, fasting blood glucose levels, insulin blood levels and fasting insulin blood levels. A patient whose glucose level and/or insulin level is determined to be higher than average for the population is then treated with a composition comprising the peptide with SEQ ID NO. 1 or its equivalent. Various treatment protocols can be used, including without limitation, oral administration and/or injections.

In some embodiment, a patient is tested for his or her fasting insulin level. If the insulin level is higher than 9 mlU/ml, the patient is treated with a composition comprising the effective amount of the peptide with SEQ ID NO. 1 and at least one excipient. In some embodiments, the effective amount is at least 2 mg per dosage. In other embodiments, the effective amount is at least 2.5 mg per dosage. In further embodiments, the effective amount is at least 3 mg per dosage. In further embodiments, the effective amount is at least 4 mg per dosage. In further embodiments, the effective amount is from 5 mg/day to 1 g/day. A dosage may be adjusted according to patient's response and tolerance level. Some patients can be treated for a period of several weeks on a daily basis. Other patients can be treated indefinitely. The patient's response to treatment can be evaluated by monitoring for stabilization of patient's glucose and/or insulin blood levels or by other methods known to a person of skill.

While not wishing to be bound by a theory, it is believed that the peptide with SEQ ID NO. 1 acts as an antagonist for pancreastatin, an endogenous peptide which is known to antagonize insulin at least by competing with the binding of insulin to the insulin receptor. The peptide with SEQ ID NO. 1 competes with pancreastatin and permits the binding of insulin to the insulin receptor, thus decreasing the amount of insulin in fasting blood and reversing the insulin resistance.

Pancreastatin (PEGKGEQEHSQQKEEEEEMAVVPQGLFRG-amide (SEQ ID NO. 2) is a chromogranin A (CgA) derived peptide and in humans it corresponds to amino acids 250-301 in CgA. The peptide with SEQ ID NO. 2 is often abbreviated as PST. The peptide with SEQ ID NO. 2 (PST) negatively regulates insulin secretion from pancreatic islets, adipokine secretion from adipocytes, as well as insulin-stimulated glucose uptake in muscle and glycogen synthesis in liver.

The peptide with SEQ ID NO. 1 (also referred to as PSTv1 or PST-NΔ3 interchangeably in this disclosure) is a variant of PST which, when administered to mice, improves glucose tolerance and insulin sensitivity in diet induced obese (DIO) mice and diabetic and obese db/db mice models. The peptide with SEQ ID NO. 1 has no adverse effects on heart function.

The peptide with SEQ ID NO. 1 is not processed from CgA in vivo. The peptide with SEQ ID NO. 1 which competes with endogenous PST action, does not cause complete inhibition of PST action. As a result, the peptide with SEQ ID NO. 1 can minimize anti-insulin action of PST when the PST level rises above the physiological level, as in the case of diabetic patients. Therefore, the side effects of the therapy with the peptide with SEQ ID NO. 1 are minimal.

The effects of the peptide with SEQ ID NO. 1 can be compared with other peptides such as insulin and adiponectin. All these peptides are administered by intramuscular injection.

Unlike the other peptides, the peptide with SEQ ID NO. 1 acts as an antagonist to anti-insulin PST and improves glucose tolerance and insulin sensitivity in diet-induced obese (DIO) C57BL/6J and obese and insulin resistant db/db mice. Molecular dynamics and docking of the peptide with SEQ ID NO. 1 with the insulin receptor reveals that the peptide with SEQ ID NO. 1 binds parallel to the αCT helix of the insulin receptor and is expected to improve insulin action.

Lack of the PST peptide with SEQ ID NO. 2 in Chga knockout (Chga-KO) mice results in increased hepatic sensitivity to insulin, which is abolished by PST supplementation.

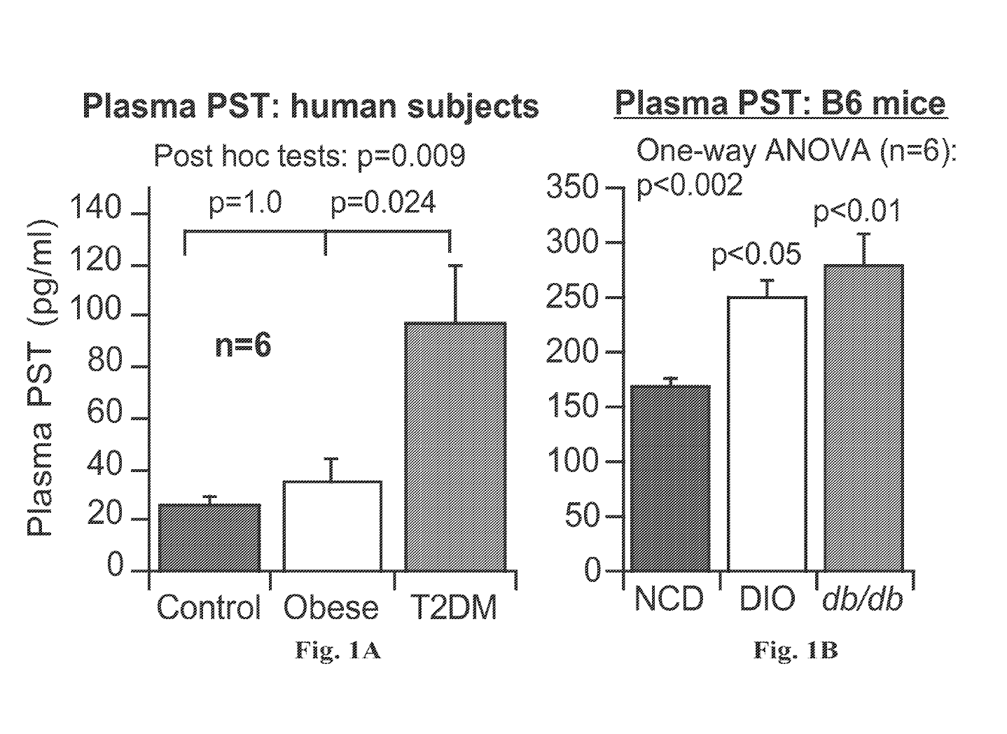

As shown in FIG. 1A, blood plasma PST is elevated ˜3.7-fold in diabetic patients. As shown in FIG. 1B, PST concentrations in diet-induced obese (DIO) or diabetic db/db mice are 50-60% higher than in control mice.

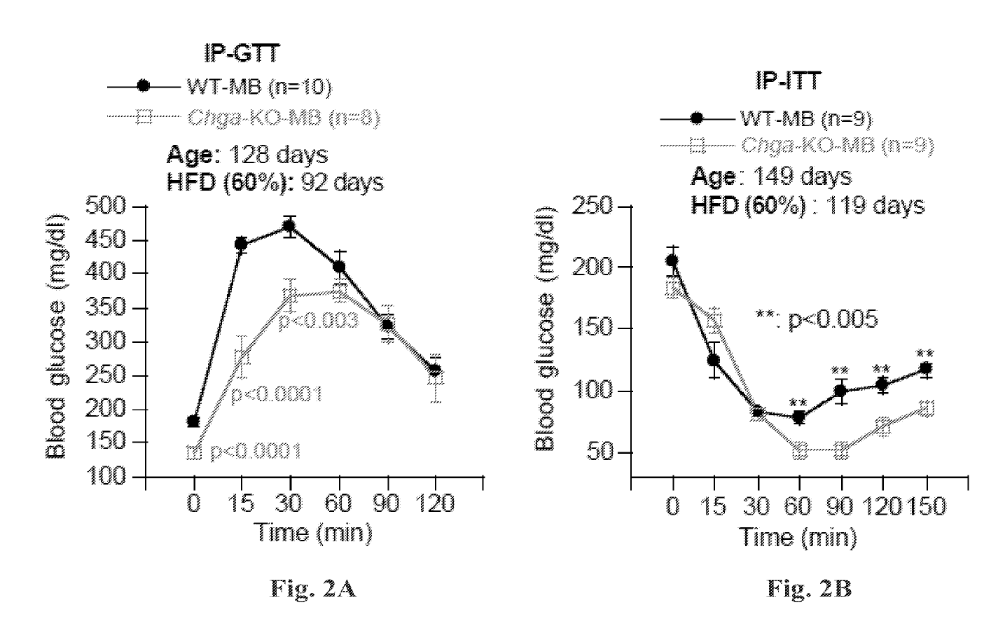

As shown in FIGS. 2A and 2B, when both WT (wild type) and PST deficient Chga-KO (chromogranin-knockout) mice with mixed genetic background were exposed to a high-fat diet for 16 weeks and their insulin sensitivity and glucose tolerance were examined, Chga-KO mice were significantly protected against insulin resistance as evidenced from glucose tolerance test (GTT), insulin tolerance test (ITT) and the increased GIR (glucose infusion rate) values in the clamp studies.

These features of Chga-KO mice are also true in mice with C57BL/6J background as shown by GTT and ITT in high fat diet fed WT and Chga-KO mice with C57BL/6J background. Administration of the PST peptide for 5 days to PST deficient Chga-KO mice on high fat diet led to hyperglycemia in Chga-KO mice similar to WT-DIO mice.

As shown in FIGS. 2A and 2B, the peptide with SEQ ID NO. 1 acts as an antagonist to PST and improves glucose tolerance (FIG. 2A) and insulin sensitivity (FIG. 2B) in the DIO model. The peptide with SEQ ID NO. 1 antagonizes persistent actions of PST when its level rises in diabetic models and suppresses the inhibitory effects of PST on insulin action.

A new pathway for PST action as defined herein may explain the mode of action of the peptide with SEQ ID NO. 1 in improving glucose homeostasis in insulin resistant and diabetic mice and may provide the basis for a strategy to treat insulin-resistant and diabetic patients.

It has been revealed that the peptide with SEQ ID NO. 1 reverses the anti-insulin like effects of PST. The peptide with SEQ ID NO. 1 causes improvement in glucose tolerance in wild type diet-induced obese (WT-DIO) mice and genetically obese Zucker rats without changing food intake or body weight.

PST acts as a negative regulator of insulin sensitivity, and its deficiency in knockout Chga-KO mice seemed to be responsible for the increased insulin sensitivity in diet induced obese knockout (KO-DIO) mice.

The treatment with the peptide with SEQ ID NO. 1 (10 μg/g/day) for 15 days lowered fasting plasma glucose levels in WT-DIO mice and obese Zucker rats, and showed improvement in GTT and ITT. While chronic PST treatment raised blood glucose level in KO-DIO mice to WT-DIO level, chronic PSTv1 (the peptide with SEQ ID NO. 1) treatment to WT-DIO did not. ITT also supported the notion that the peptide with SEQ ID NO. 1 has a hypoglycemic effect possibly by competing against endogenous PST.

Acute PST treatment induces hyperglycemia in insulin-sensitive Chga-KO mice on a normal chow diet. Chronic PST supplementation to both WT and Chga-KO mice on a normal chow diet (NCD) induces insulin resistance. The peptide with SEQ ID NO. 1 competes with PST and prevents PST-induced hyperglycemia. In NCD-fed Chga-KO mice (KO-NCD), the hyperglycemic effect of PST effect is attenuated by concomitant acute injection of PSTv1 (the peptide with SEQ ID NO. 1). Moreover, chronic supplementation of the peptide with SEQ ID NO. 1 reverses chronic effects of PST treatment. The treatment with the peptide with SEQ ID NO. 1 alone has no effect, presumably because Chga-KO mice lacks endogenous PST for PSTv1 (the peptide with SEQ ID NO. 1) to compete with.

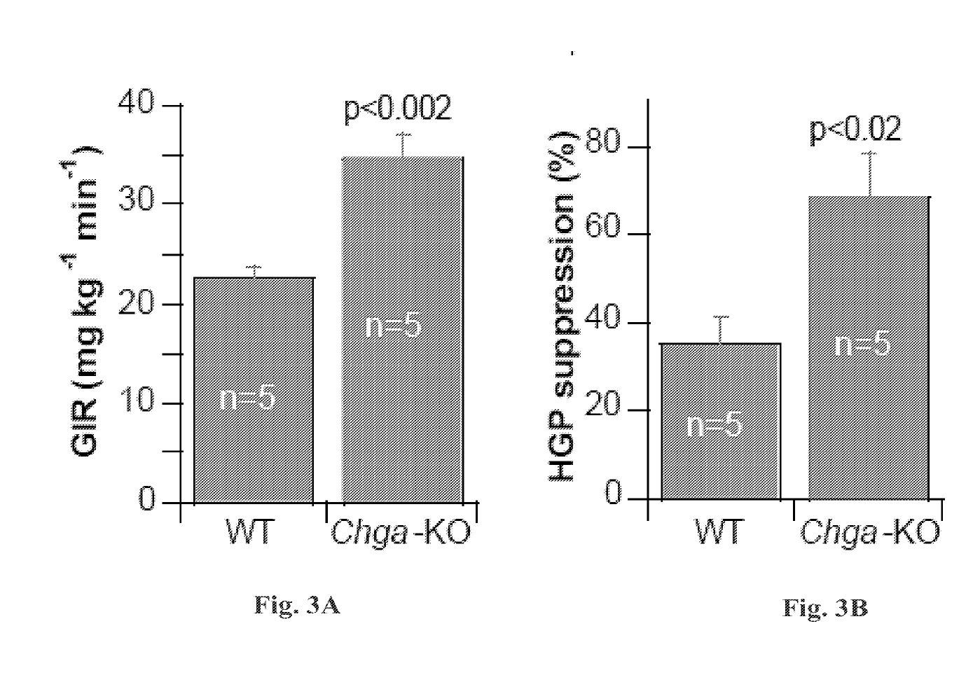

As shown in FIGS. 3A and 3B, the peptide with SEQ ID NO. 1 improves the insulin sensitivity in WT-DIO mice. As shown in FIG. 3A, hyperinsulinemic-euglycemic clamp studies revealed significant improvement in glucose infusion rate (GIR; FIG. 3A) and % suppression of hepatic glucose production (HGP; FIG. 3B) in the group treated with the peptide with SEQ ID NO. 1 (PSTv1) as well as the insulin stimulated glucose disposal rate (IS-GDR) without significant decrease in weight of liver and adipose tissues. Improvement in GIR indicates hepatic target of PSTv1 (the peptide with SEQ ID NO. 1) action. Decreased NEFA levels after PSTv1 (the peptide with SEQ ID NO. 1) treatment indicates improved insulin sensitivity in the adipose tissue.

In further embodiments, PSTv1 (the peptide with SEQ ID NO. 1) modulates fasting insulin level and glucose-stimulated insulin secretion. In WT-DIO mice treated with PSTv1 (the peptide with SEQ ID NO. 1), the inventors have discovered a concomitant decrease in plasma insulin and glucose concentrations without a change in insulin/C-peptide molar ratio suggesting that PSTv1 (the peptide with SEQ ID NO. 1) improved insulin sensitivity.

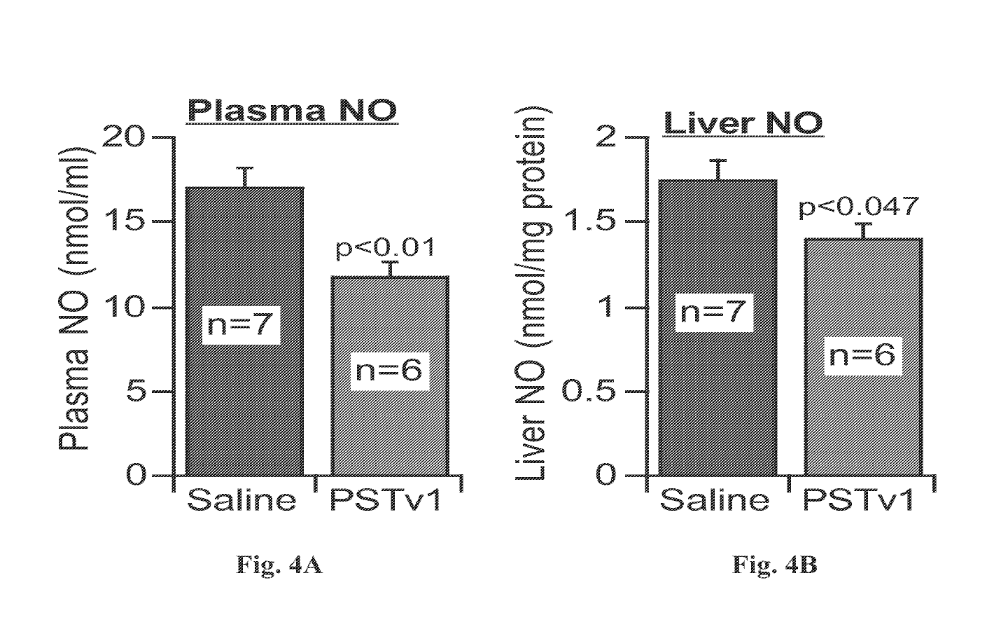

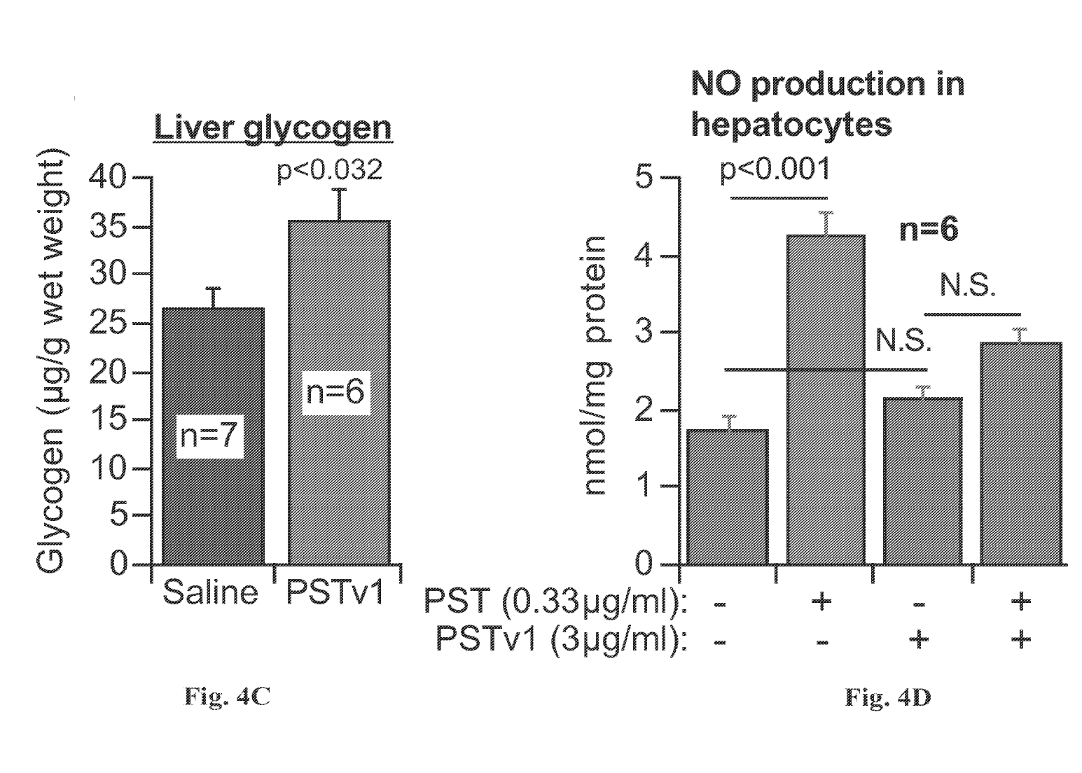

As shown in FIGS. 4A-4D, to compare the effects of PSTv1 (the peptide with SEQ ID NO. 1) vs. PST on glucose-stimulated insulin secretion (GSIS), the inventors analyzed plasma insulin levels in Chga-KO mice during GTT experiments. The PST treatment lowers GSIS without lowering plasma glucose concentration indicating insulin resistance whereas, PSTv1 (the peptide with SEQ ID NO. 1) lowers glucose concentrations without affecting GSIS. Moreover, PSTv1 (the peptide with SEQ ID NO. 1) pretreatment partially reverses the decline in GSIS caused by PST.

As shown in FIGS. 4A and 4B, PSTv1 (the peptide with SEQ ID NO. 1) effects plasma nitric oxide (NO) and liver nitric oxide (NO). As shown in FIGS. 4C and 4D, it also affects liver glycogen content in WT-DIO mice and PST-induced NO production in hepatocytes. PST PSTv1 (the peptide with SEQ ID NO. 1) decreased NO in both plasma and liver of DIO mice (FIG. 4A and FIG. 4B) supporting the proposed antagonism between PST and PSTv1 (the peptide with SEQ ID NO. 1).

This was further demonstrated in cultured hepatocytes, where PST-stimulated NO production was effectively blocked by co-treatment with PSTv1 (the peptide with SEQ ID NO. 1). As shown in FIG. 4C and consistent with the negative regulation of glycogen production by NO, PSTv1 (the peptide with SEQ ID NO. 1) increases liver glycogen in WT-DIO mice.

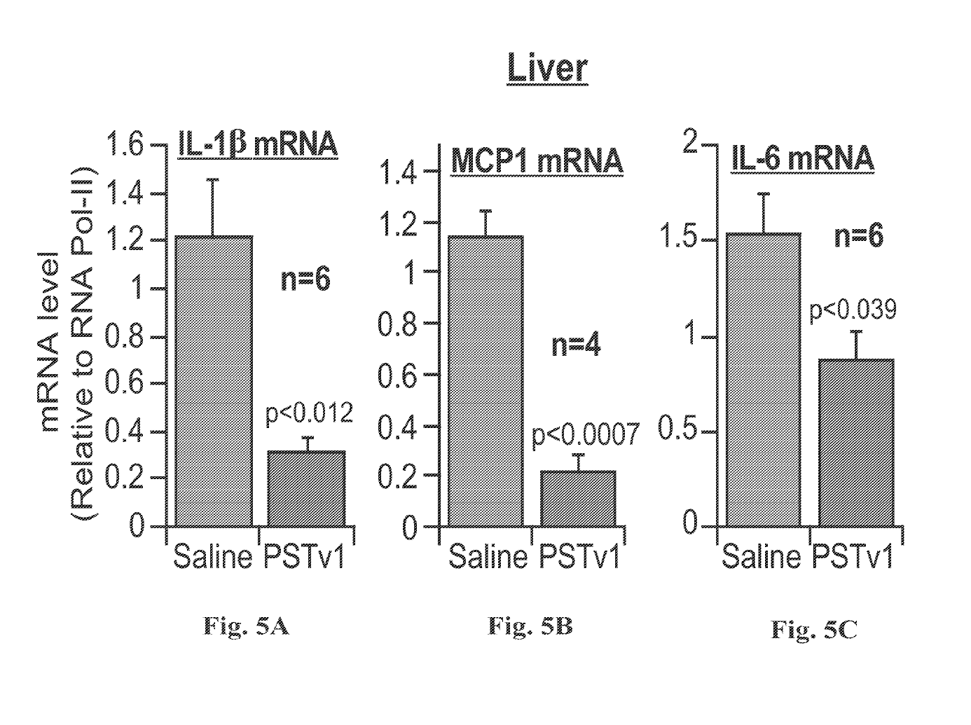

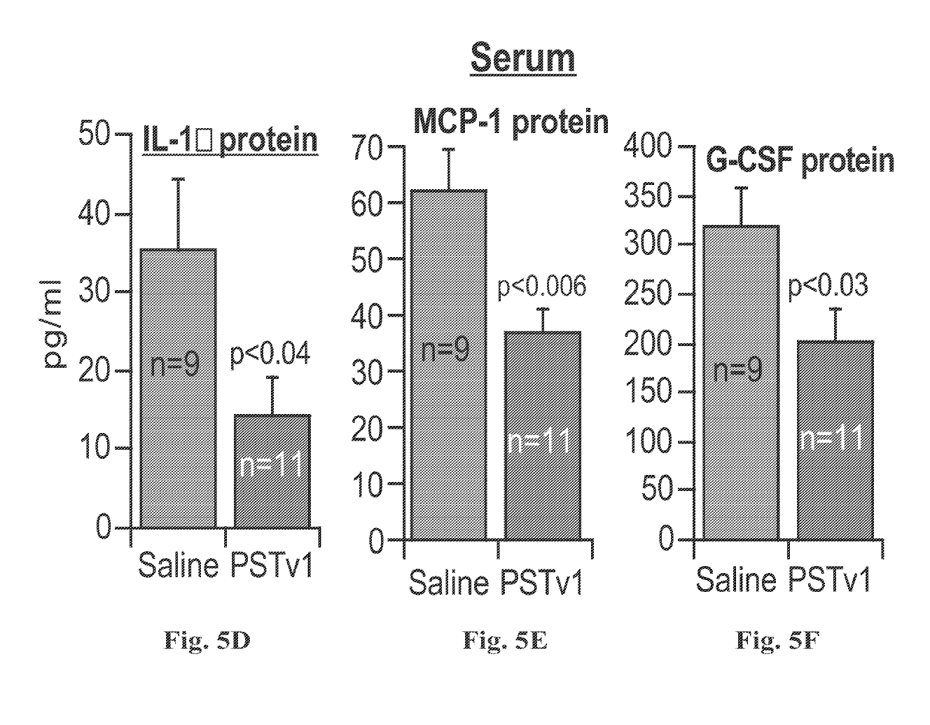

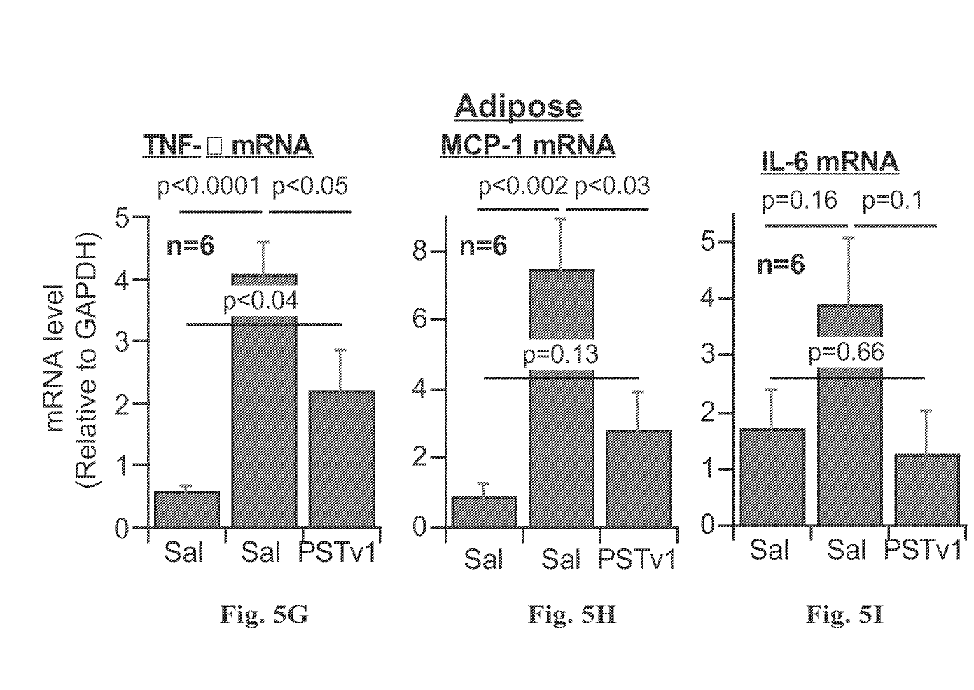

As shown in FIGS. 5A-5I, PSTv1 (the peptide with SEQ ID NO. 1) suppresses inflammation and regulates expression of cytokine mRNA and protein. PST-deficient insulin sensitive Chga-KO mice demonstrates low plasma cytokine levels. Anti-PST effects of PSTv1 (the peptide with SEQ ID NO. 1) decreases inflammatory cytokine levels in DIO mice. In fact, PSTv1 (the peptide with SEQ ID NO. 1) suppresses expressions of interleukin (IL-1β and IL-6) and monocyte chemoattractant protein (MCP-1) genes in liver as well as plasma MCP-1, IL-1β and granulocyte colony stimulating factor (G-CSF) proteins in WT-DIO mice. As further shown in FIGS. 5G-5I, PSTv1 (the peptide with SEQ ID NO. 1) also attenuates expression of TNF-α, MCP-1 and IL-6 in adipose tissue of WT-DIO mice.

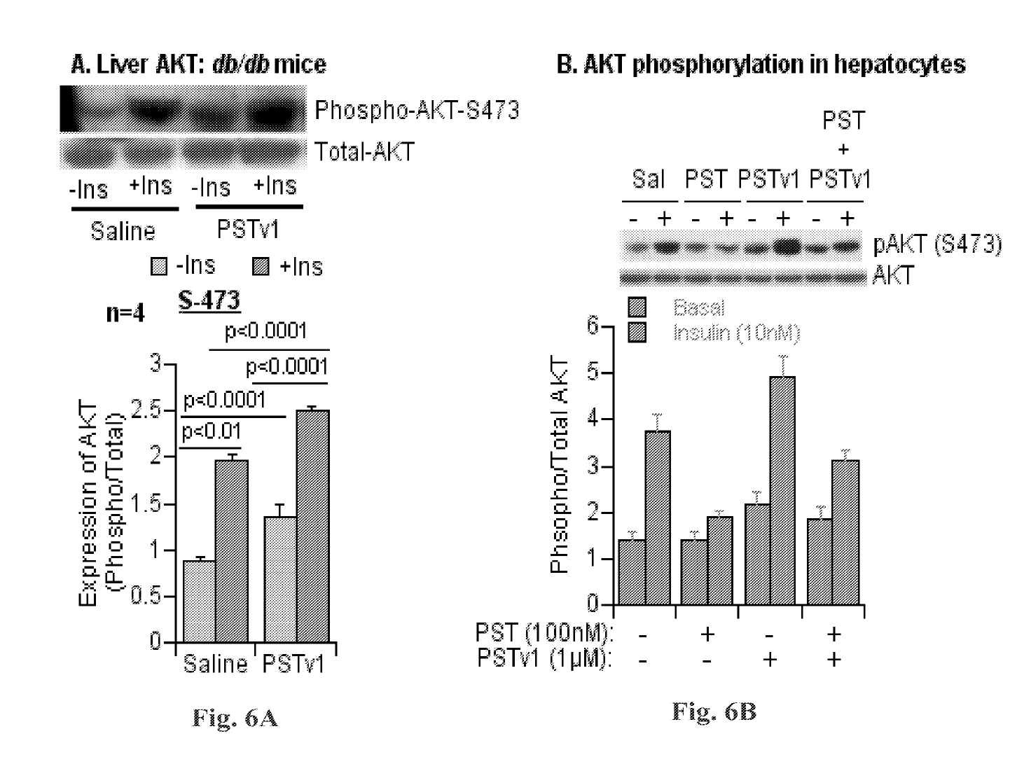

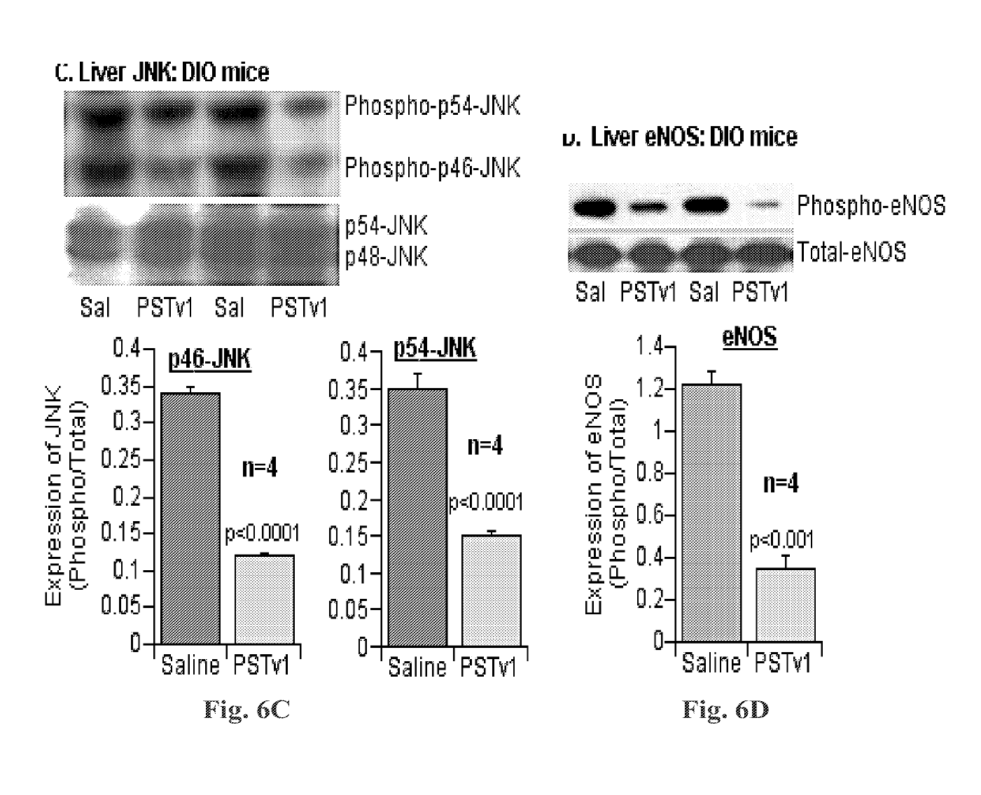

In some embodiments, the peptide with SEQ ID NO. 1 can be used in combination with insulin in order to potentiate the effect of insulin treatment. As shown in FIGS. 6A-6D, in both db/db and WT-DIO mice, chronic PSTv1 (the peptide with SEQ ID NO. 1) treatment (for 14 days) potentiated the acute effect of insulin treatment (3 min) on AKT phosphorylation in liver (from db/db and WT-DIO mice) and muscle (from WT-DIO mice) suggesting sensitization of insulin signaling pathway by PSTv1 (the peptide with SEQ ID NO. 1) in insulin resistant mice.

PSTv1 (the peptide with SEQ ID NO. 1) also stimulates phosphorylation of GSK-3β in the DIO liver. PSTv1 (the peptide with SEQ ID NO. 1) alone stimulates Akt phosphorylation in both models raising the possibilities that either inhibition of a phosphatase activity (such as PTEN) or increased bioavailability of PI, P2 (a substrate for PI-3-kinase) by PSTv1 (the peptide with SEQ ID NO. 1) might have contributed to the increase in Akt phosphorylation signal. In contrast to pAKT signaling in WT-DIO mice, phosphorylation of hepatic endothelial nitric oxide synthase (eNOS) is suppressed by the PSTv1 (the peptide with SEQ ID NO. 1) treatment because PSTv1 (the peptide with SEQ ID NO. 1), unlike PST, suppresses NO production and promotes glycogen storage.

As shown in FIGS. 6A-6D, suppression of phospho-c-JUN-N terminal kinase (pJNK) signals in liver, the decrease in plasma MCP-1 and IL-1β concentrations, downregulation of IL-1β and IL-6 gene expressions in liver and TNF-α and MCP-1 expressions in the adipose tissue suggest an anti-inflammatory effect of PSTv1 (the peptide with SEQ ID NO. 1). Overall, PSTv1 (the peptide with SEQ ID NO. 1) appears to be anti-inflammatory and supportive of insulin action.

PST interacts with ER chaperone, GRP78/BiP. In a pull down assay using biotinylated PST, the inventors found that PST binds to endoplasmic reticulum (ER) Chaperone, Bip/GRP78 and expression of BiP/GRP78 is elevated in liver of Chga-KO mice. Moreover, PST inhibits GRP78's ATPase enzymatic activity, and impairs its biosynthetic response to UPR activation. PST as an inhibitor of ATPase activity augments expression of gluconeogenic gene and G6Pase. GRP78 over-expression antagonizes this PST action.

Without wishing to be bound by this theory, the inventors conclude that (i) a part of ER chaperone GRP78 is located at the cell surface and the rest in the ER lumen, and (ii) PST interacts with GRP78 at the cell surface which, in turn, is internalized via endocytosis. The interaction between PST and GRP78 on cell surface and its subsequent internalization contributes to insulin resistance. (iii) PSTv1 (the peptide with SEQ ID NO. 1) prevents binding to GRP78 and internalization of PST. From that point of view, PSTv1 (the peptide with SEQ ID NO. 1) may improve insulin action by preventing interactions between PST and GRP78. In summary, PSTv1 (the peptide with SEQ ID NO. 1) improves insulin sensitivity in part by reducing (i) inflammation and (ii) ER stress.

In further embodiments, the peptide with SEQ ID NO. 1 can be chemically modified to improve its action and increase its half-life. In some embodiments, PSTv1 (the peptide with SEQ ID NO. 1) is composed of L-amino acids. A retro-inverso version (RT-PSTv1, the peptide with SEQ ID NO. 1) can be synthesized with D-amino acids. RT-PSTv1 (the peptide with SEQ ID NO. 1) is functionally active with added advantage of being resistant to proteolytic degradation when injected in vivo.

PST-deficient Chga-KO mice, when raised on high fat diet (HFD), become obese but maintain insulin sensitivity (in contrast to the WT mice). If the PST level is reduced or if a peptide competes with PST action, it might reverse insulin resistance and improve glucose tolerance in HFD fed WT-DIO mice.

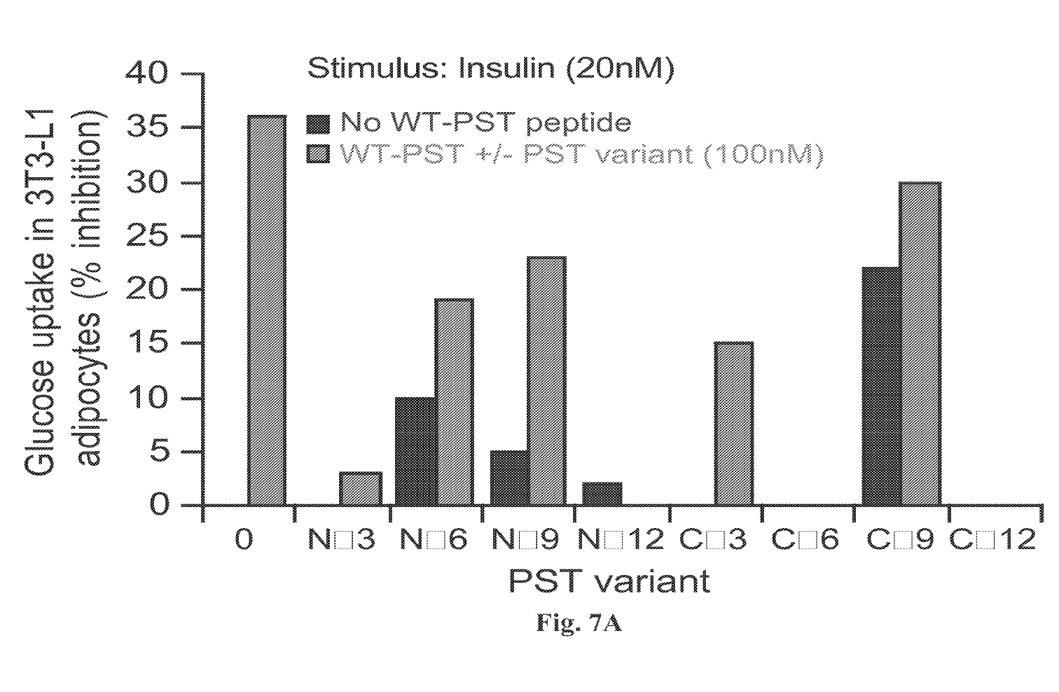

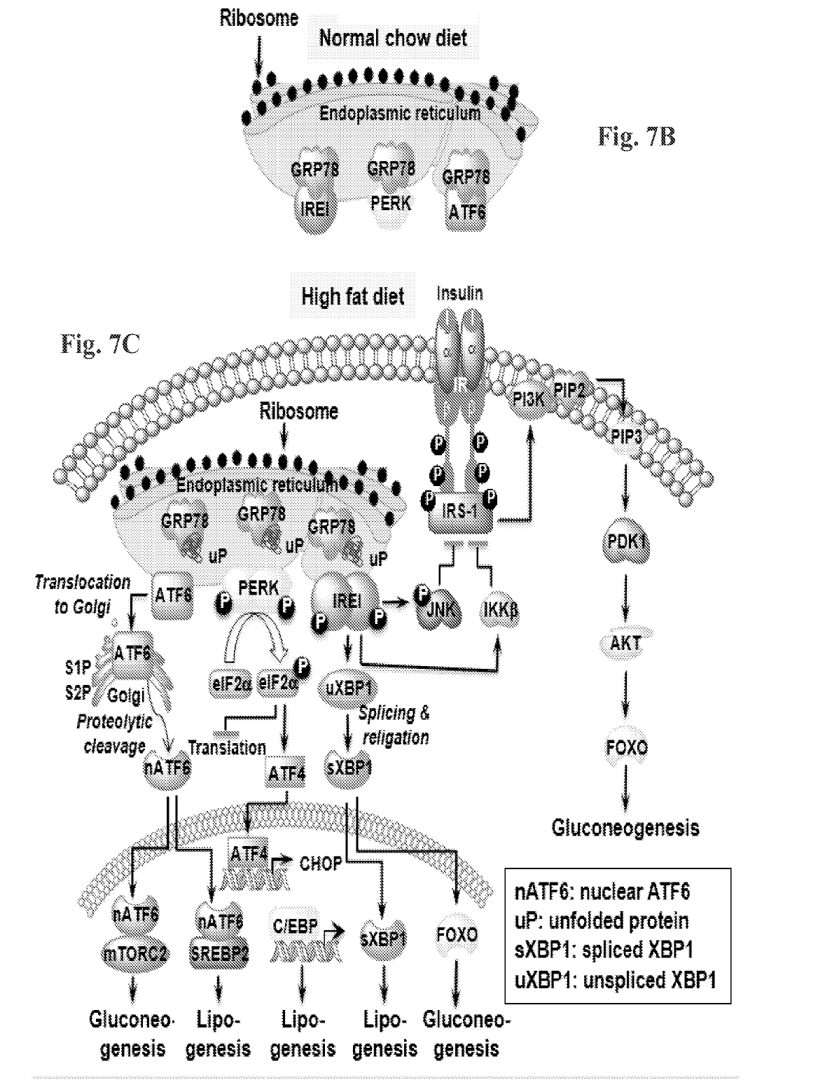

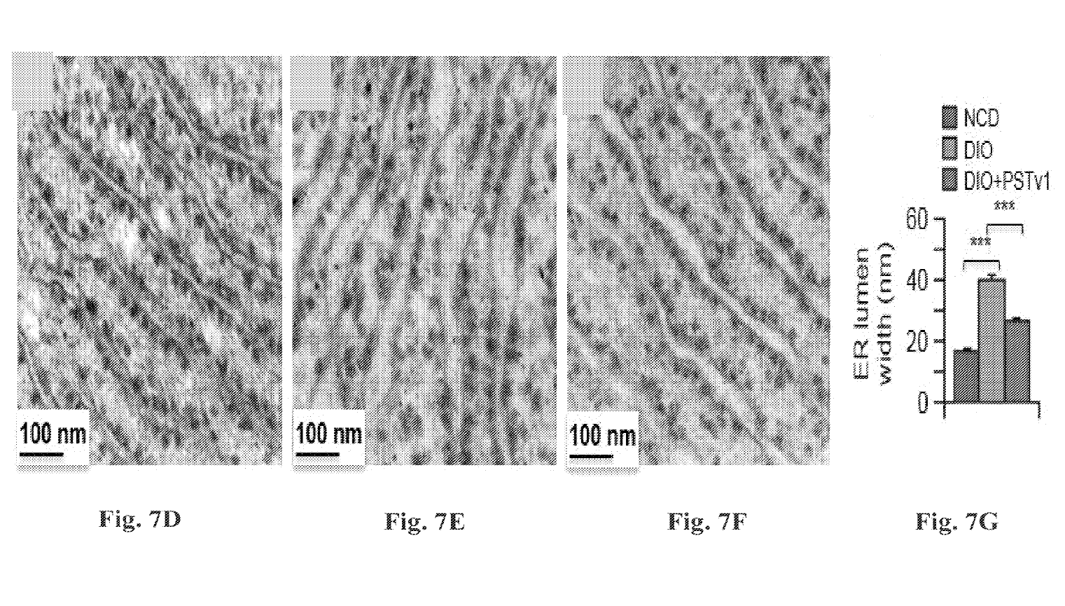

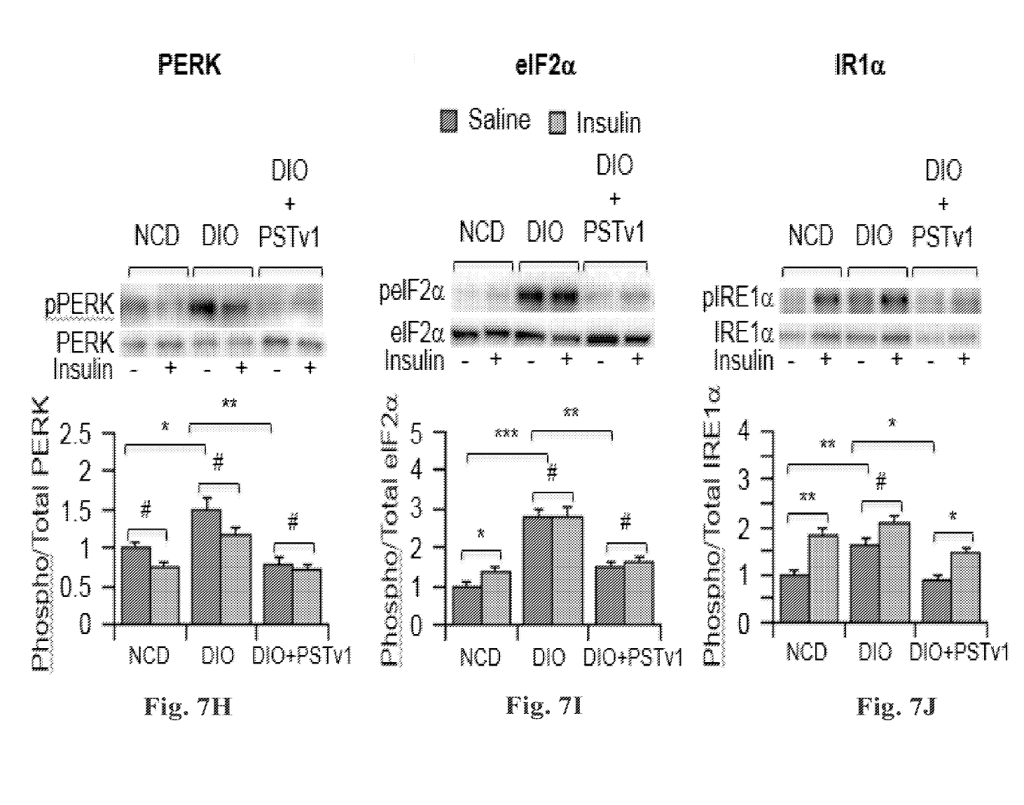

As shown in FIG. 7A, various PST variant peptides have been analyzed for their ability to reverse insulin resistance. Mutant PST peptides were synthesized and screened. The peptides were synthesized as various deletion peptides of PST. Native PST (SEQ ID NO. 2) inhibited insulin-stimulated glucose uptake and leptin production in adipocytes. The inventors tested various deletion mutant PST peptides whether one or more of them would compete and prevent native PST action in adipocytes. As shown in FIG. 7A, without any PST variant present, PST inhibited 35% of insulin-stimulated glucose uptake in 3T3-L1 adipocytes in culture (the bar with “0” PST variant). Surprisingly, the presence of PST variant NΔ3 (100 nM), also called PSTv1 (the peptide with SEQ ID NO. 1), with PST prevented the inhibition by PST effectively.

These results support the claim that PSTv1 (the peptide with SEQ ID NO. 1) may interfere with PST action and protect insulin action.

As shown in FIG. 7C in comparison to FIG. 7B, high-fat diet (HFD) induces endoplasmic reticulum (ER) stress and decreases insulin sensitivity. As shown in FIG. 7B, the ER is essential for the folding and maturation of proteins, lipid biosynthesis, and homeostasis of calcium and redox potential. The accumulation of unfolded and misfolded proteins in the ER lumen, termed ER stress, leads to activation of signaling pathways to counteract defects in protein folding. This unfolded protein response (UPR) increases the repair activities, reduces global protein synthesis, and activates ER-associated protein degradation (ERAD). UPR is mediated by three major branches of signaling (as shown in Scheme 1); (i) protein kinase RNA-like ER kinase (PERK)-mediated, (ii) inositol-requiring kinase I (IRE1)-mediated, and (iii) activating transcription factor 6 (ATF6)-mediated.

As shown in FIG. 7C, if ER stress becomes chronic as seen under the conditions of high-fat diet, UPR cannot cope up with the repair demands, protein-folding homeostasis breaks down, leading to activation of apoptotic pathway. Thus, ER stress and the UPR play important roles in the pathogenesis of multiple human metabolic diseases such as insulin resistance (IR), diabetes, obesity, non-alcoholic fatty liver disease, and atherosclerosis.

FIG. 7C shows how defective protein folding causes ER stress and evokes UPR. Three ER-resident transmembrane proteins have been identified as sensors of ER stress: IREI, PERK and ATF6. Dissociation of GRP78 binding with UPR and binding with unfolded proteins (uP) are notable. UPRs' direct regulation of gluconeogenesis and lipogenesis and indirect regulation of gluconeogenesis by inhibiting IRS signaling are also notable.

Although the ER stress activates all the three UPR branches of the UPR signaling simultaneously, the behavior of each of these branches varies markedly in time after the onset of the stress.

As shown in FIGS. 7D-7G, the ER regulates synthesis, folding and transport of Golgi, lysosomal, secretory and cell surface proteins. The ER also controls synthesis of N-linked oligosaccharides and the first steps of N-glycosylation and anchoring of glycosyl phosphatidyl inositol. In addition, the ER is also involved in calcium storage, lipid metabolism and drug detoxification in liver. When the folding capacity of the ER is overwhelmed owing to an increase in protein load and/or disruption of the folding capacity it results in the development of ER stress. As a dynamic organelle, the ER responds to ER stress by increasing the amount of ER membrane. As shown in FIGS. 7D, 7E and 7G, ultrastructural analyses of the liver revealed increased ER dilation in liver of HFD-induced obese (DIO) mice compared to NCD (normal chow diet) fed mice. As shown in FIGS. 7F and 7G, treatment of DIO mice with PSTv1 (the peptide with SEQ ID NO. 1) decreases ER lumen width, indicating attenuation of ER stress by PSTv1 (the peptide with SEQ ID NO. 1).

As shown in FIGS. 7H and 7I, PSTv1 (the peptide with SEQ ID NO. 1) suppresses phosphorylation of PERK and eLF2α in diet-induced obese mice. The PERK pathway is the first to be activated followed by the activation of the ATF6 and the IREI pathways, respectively. PERK is a type I serine threonine transmembrane kinase. In response to ER stress, PERK is released from ER chaperone, GRP78/BiP, dimerizes and promotes its autophosphorylation and activation. Activated PERK reduces the general cap- or elF2α-dependent translation by phosphorylating serine-51 of the α subunit of elF2. As shown in FIG. 7H, PERK phosphorylation was increased in the DIO liver, but it was suppressed by PSTv1 (the peptide with SEQ ID NO. 1) treatment. As shown in FIG. 7I, elF-2α phosphorylation was increased in the DIO liver, but it was suppressed by PSTv1 (the peptide with SEQ ID NO. 1) treatment.

As shown in FIG. 7J, PSTv1 (the peptide with SEQ ID NO. 1) decreases the IRE1 arm of the UPR pathway. IRE1α is a transmembrane protein that consists of an N-terminal luminal sensor domain, a single transmembrane domain and C-terminal cytosolic effector containing a Ser/Thr kinase domain and an endoribonuclease domain. Xbp1 mRNA is the substrate of the IREI endonuclease domain. Besides acting on Xbp1, IREIα also controls its own expression by cleaving its own mRNA. Xbp1 decreases gluconeogenesis by inducing degradation of FOXO1. Increased phosphorylation of IRE1α in DIO mice as compared to NCD mice indicates activation of the IRE1α arm of the UPR. As shown in FIG. 7J, PSTv1 (the peptide with SEQ ID NO. 1) decreases HFD-induced phosphorylation of IRE1α. PSTv1 treatment did not alter ATF6 signaling significantly.

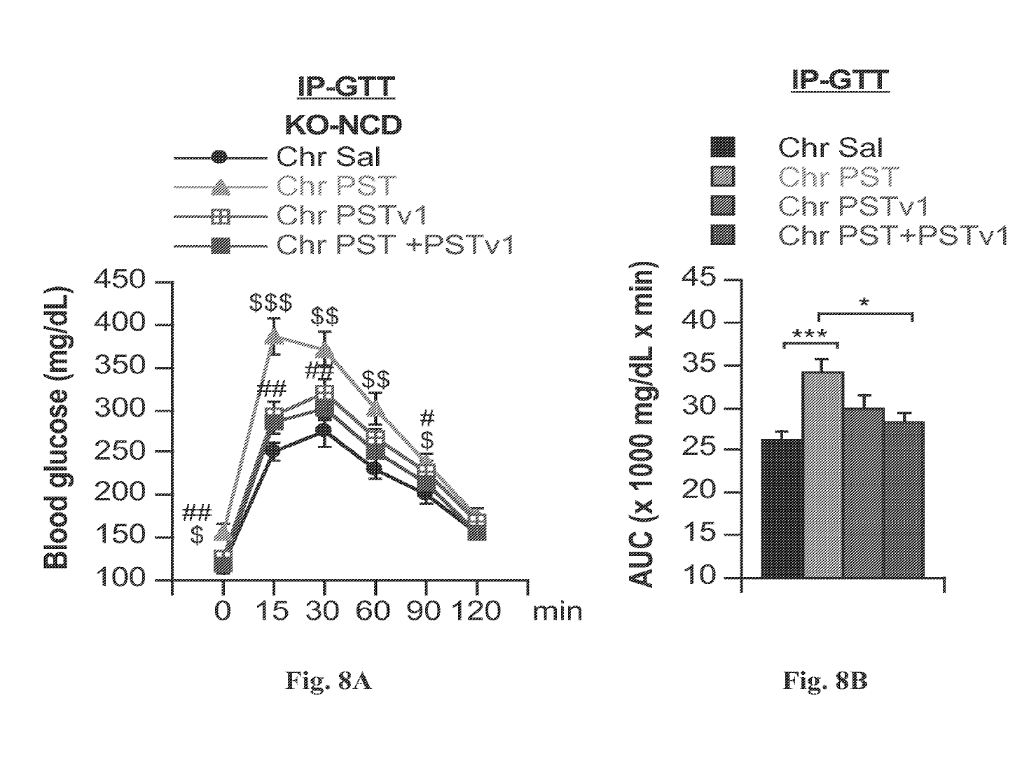

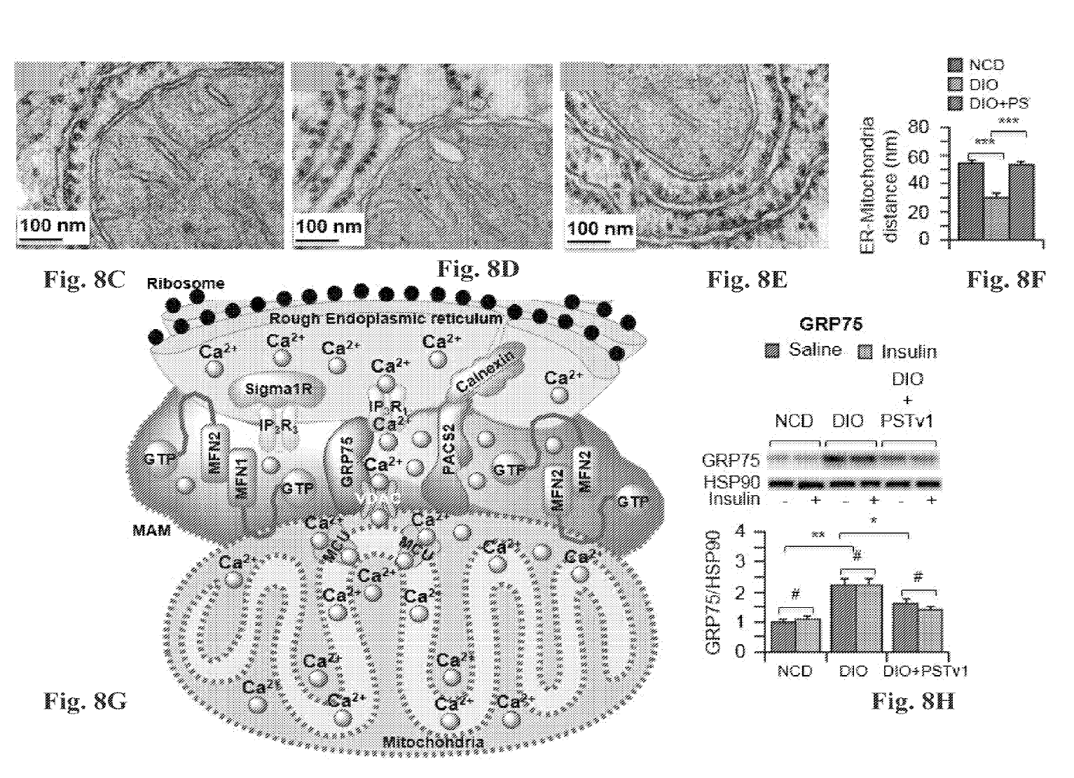

As shown in FIGS. 8A and 8B, the effects of PST, much like anti-insulin, were reversed by a PST-variant peptide, PSTv1 (the peptide with SEQ ID NO. 1). To combat anti-insulin like effects of PST in vivo, PSTv1 (the peptide with SEQ ID NO. 1) inhibits competitively PST and improves glucose intolerance in mice rendered insulin resistant. The inventors used Chga-KO mice to remove endogenous PST in the background. These mice are very insulin sensitive but when treated with PST, they become hyperglycemic and glucose intolerant. As shown in FIGS. 8A and 8B, when GTT were conducted in normal chow-fed Chga-KO mice (KO-NCD), the hyperglycemic effect of chronic PST treatment is attenuated by chronic treatment with PSTv1 (the peptide with SEQ ID NO. 1). PSTv1 (the peptide with SEQ ID NO. 1) alone has no effect on Chga-KO mice, presumably because of the lack of endogenous PST with which PSTv1 (the peptide with SEQ ID NO. 1) could compete.

To further establish the insulin sensitizing effects of PSTv1 (the peptide with SEQ ID NO. 1), obese and insulin resistant WT-DIO mice were treated with PSTv1 (the peptide with SEQ ID NO. 1) for 15 days. Subsequent GTT and ITT showed lower fasting plasma glucose levels as well as improved glucose tolerance and insulin sensitivity (see FIGS. 3A and 3B) possibly by competing against endogenous PST. While chronic PST supplementation to both WT and Chga-KO mice on a normal chow diet induced insulin resistance, chronic PSTv1 (the peptide with SEQ ID NO. 1) improves insulin sensitivity. These results demonstrate the PST deficiency as the primary reason for insulin sensitivity and protection against diet-induced insulin resistance in Chga-KO mice.

As shown in FIGS. 8C-8H, PSTv1 (the peptide with SEQ ID NO. 1) decreases expression of mitochondria-associated membrane (MAM) and matrix protein, GRP75 in DIO liver. A hallmark of ER stressed condition in DIO liver is the close proximity between ER and mitochondria leading to increased formation of MAM containing glucose regulated protein 75 (GRP75), phosphofurin acidic cluster protein 2 (PACS2), and mitofusin 1 and 2 (MFN1 & 2) as the bridging members between these two organelles as shown in FIG. 8G.

PSTv1 (the peptide with SEQ ID NO. 1) treatment prevents the closeness between ER and mitochondria and increases the gap as shown in micrographs of FIGS. 8C, 8D and 8E, where the juxtaposition of ER and mitochondria is shown by arrows, and in calculations of FIG. 8F. At the same time, PSTv1 (the peptide with SEQ ID NO. 1) decreases the expression of MAM protein, GRP75 in DIO liver, as shown in FIG. 8H. The importance of GRP75 is that it is part of the MAM, which is crucial for calcium transport to mitochondria. It appears that ER stress in DIO liver might have caused more interactions between ER and mitochondria leading to increased calcium and lipid entry into mitochondria and mitochondrial stress. PSTv1 (the peptide with SEQ ID NO. 1) prevents the interaction between ER and mitochondria.

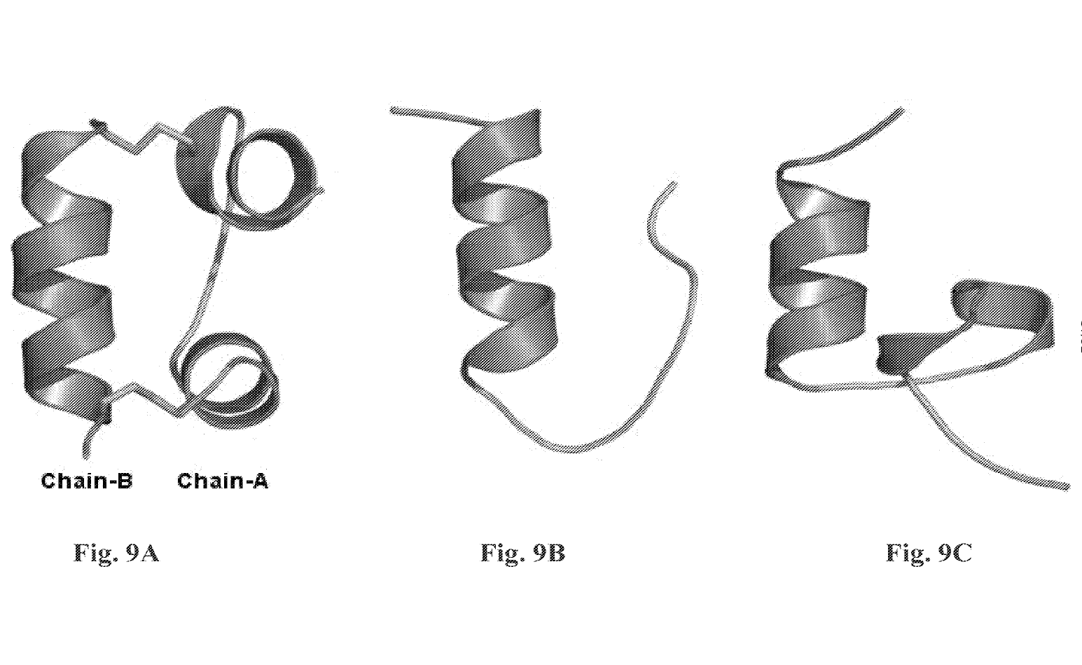

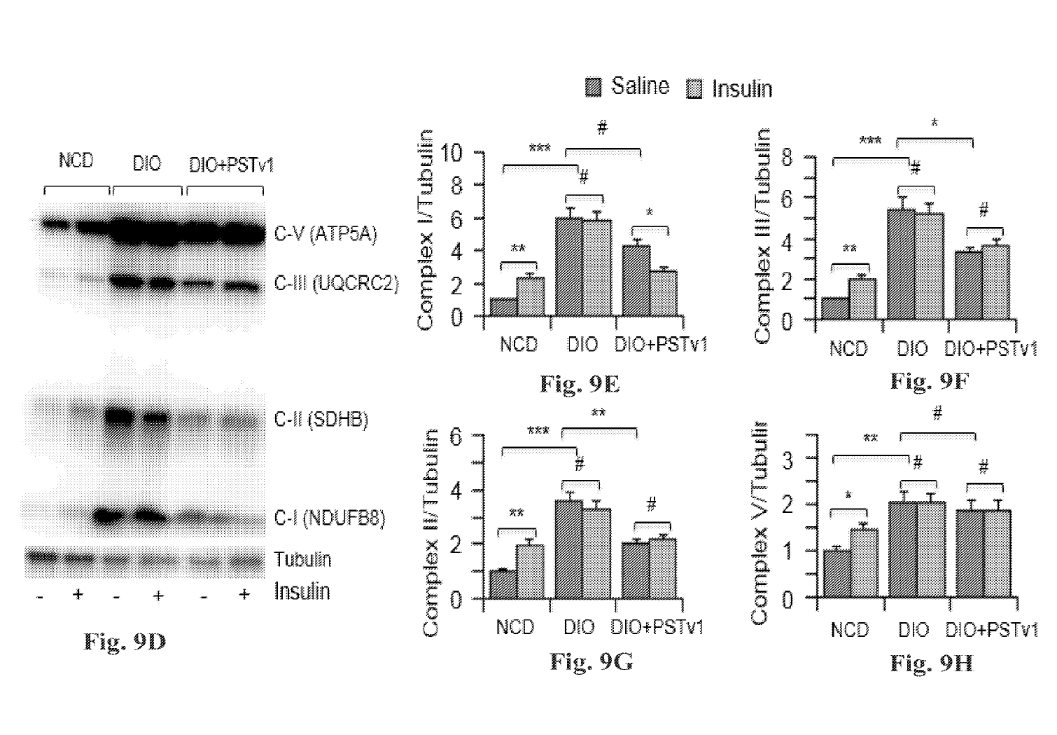

As shown in FIGS. 9A-9C, the peptide with SEQ ID NO. 1 mimics insulin structure. As can be seen from FIG. 9A, the structure of insulin is obtained from the crystal structure of insulin-bound insulin receptor, as available in protein data bank (PDB ID: 3W11). Insulin contains two chains, chain A and B that are bridged by two disulfide bonds (see FIG. 9A). While chain B is comprised of a long α-helix, chain A contains two small 3₁₀ helices connected by a loop. The structures of wild type PST (PST-WT) and its variant, PST-NΔ3 (the peptide with SEQ ID NO. 1) are obtained from large-scale molecular dynamics (MD) simulations.

FIG. 9B depicts the structure of wild type PST. FIG. 9C depicts the structure of the peptide with SEQ ID NO. 1. As shown in FIGS. 9A and 9B, both PST structures are seen to contain significant helicity. A structural comparison between the three peptides in FIGS. 9A, 9B and 9C shows that both wild-type and the PST variant with SEQ ID NO. 1 mimic insulin structure. The α-helix of PST-WT resembles the insulin chain B. Moreover, the extended 3₁₀-helix present in this peptide also mimics the lower half of insulin chain A. The structure of PST-NΔ3 (the peptide with SEQ ID NO. 1), on the other hand, resembles even more closely to insulin chain B, in which the length of the α-helix is very comparable to insulin chain B. The extent of helicity in PST-NΔ3 is, thus, larger than PST-WT and very similar to the helicity present in insulin. The 3D structure of insulin is obtained from crystal structure. Time-averaged structures of PST-WT and the PST-NΔ3 variant are obtained from individual MD simulation of 400 ns long. Length of the chain B α-helix of insulin and PST-NΔ3 variant is very similar. The α-helix present in PST-WT is smaller than insulin chain B, but space occupied by the base of PST-WT is very similar to insulin.

As shown in FIGS. 9D-9I, PSTv1 (the peptide with SEQ ID NO. 1) attenuates hyperactivity of mitochondrial respiratory chain complexes. In DIO liver, mitochondrial respiratory chain complexes I, II, III and V are overexpressed as shown in FIGS. 9D-9F, suggesting increased oxidation and production of reactive oxygen species (ROS) leading to damage to cellular functions. PSTv1 (the peptide with SEQ ID NO. 1) treatment attenuates expression of specifically complex II and III, as shown in FIGS. 9G and 9H. This suppresses ROS production and alleviates mitochondrial dysfunction.

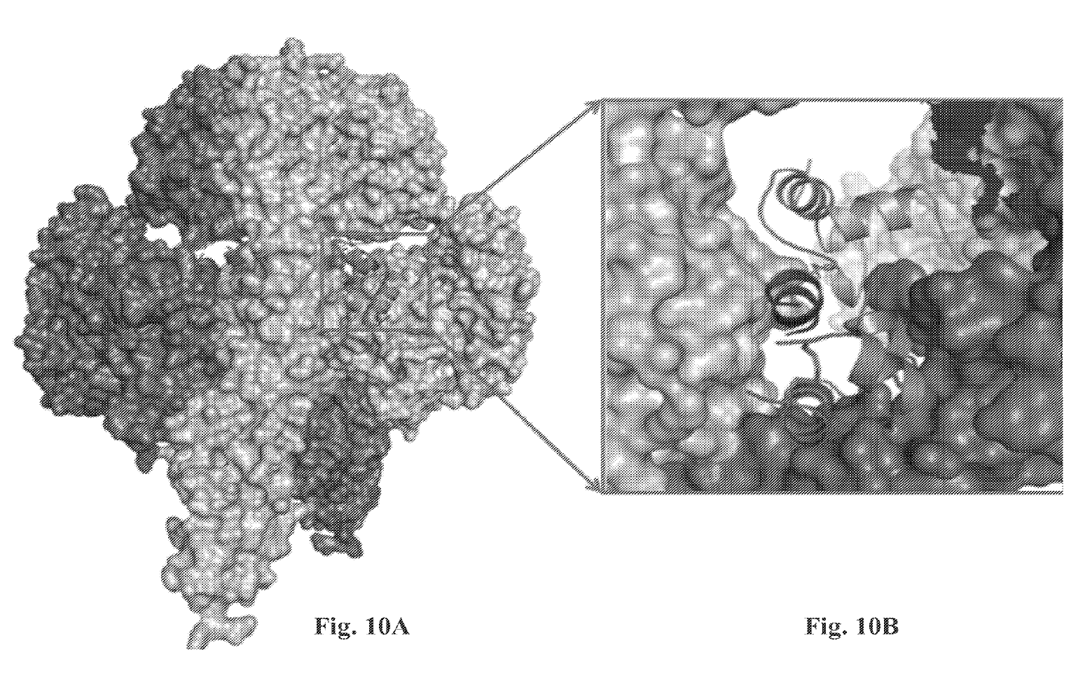

Without wishing to be bound by a theory, the inventors conclude that the peptide with SEQ ID NO. 1 interacts with the insulin receptor as shown in the models of FIGS. 10A and 10B. Noting the structural similarities between insulin and PSTs, the inventors explored the binding of the peptide with SEQ ID NO. 1 to the insulin receptor. For this purpose, protein-protein docking was attempted, in which both PST-WT (the peptide with SEQ ID NO. 2) and PST-NΔ3 (the peptide with SEQ ID NO. 1) were allowed to sample the entire insulin receptor space independently (blind docking, i.e. without any bias on binding pocket).

The docking was performed with Fast Fourier Transform based docking algorithm, ZDOCK, whose scoring function relies on the calculations of pairwise shape complementarity, desolvation and electrostatics interactions of the two partner proteins. As a control, the crystal conformation of extracted insulin was allowed to sample the entire receptor space, using the same protocol as followed for PST.

Chromogranin A knockout (Chga-KO) mice exhibit enhanced insulin sensitivity despite obesity. On a high fat diet (HFD), Chga-KO mice (KO-DIO) remain more insulin sensitive than wild-type DIO (WT-DIO) mice. Concomitant with this phenotype is enhanced Akt and AMPK signaling in muscle and white adipose tissue (WAT) as well as increased FoxO1 phosphorylation and expression of mature Srebp-1c in liver and downregulation of the hepatic gluconeogenic genes, Pepck and G6pase.

KO-DIO mice also exhibited downregulation of cytokines and pro-inflammatory genes and upregulation of anti-inflammatory genes in WAT, and peritoneal macrophages from KO mice displayed similarly reduced pro-inflammatory gene expression. The insulin-sensitive, anti-inflammatory phenotype of KO-DIO mice is masked by supplementing PST. Conversely, a PST variant peptide PSTv1 (PST-NΔ3: CHGA₂₇₆₋₃₀₁, the peptide with SEQ ID NO. 1), lacking PST activity, simulated the KO phenotype by sensitizing WT-DIO mice to insulin. In summary, the reduced inflammation due to PST deficiency prevented the development of insulin resistance in KO-DIO mice. Thus, obesity manifests insulin resistance only in the presence of PST, and in its absence obesity is dissociated from insulin resistance.

Insulin was found to dock in the crystal structure position of the receptor (i.e. docked complex and insulin-bound insulin receptor crystal structure were very similar), thus validating this docking protocol. Both in the docking complex and crystal structure, it was found that major motive of binding of insulin to the receptor is through the parallel stacking of chain B of insulin with the αCT helix of the receptor (see FIGS. 10A and 10B).

From the docking studies of PST-WT to the insulin receptor, it was found that PST binds to the insulin binding pocket. This is shown in FIG. 10A. The variant PST-NΔ3 (the peptide with SEQ ID NO. 1) also binds to the insulin binding site (FIG. 10A). However, their binding patterns are different. While the α-helix of PST-WT binds perpendicular to the αCT helix of the receptor (helix 2 in FIG. 10B), the long α-helix of PST-NΔ3 variant (the peptide with SEQ ID NO. 1) orients parallel to the αCT helix of the receptor (helix 3 in FIG. 10B). Insulin is shown as helix 1, while the αCT helix of the insulin receptor is shown as helix 4.

It is believed that PST-WT blocks the insulin binding to the insulin receptor. For the peptide with SEQ ID NO. 1, however, the similar mode of binding to that of the insulin chain B [i.e. parallel stacking of variant (helix 3 in FIG. 10B) and insulin α-helix (helix 1 in FIG. 10B) with αCT helix of IR (helix 4)], can maintain insulin signaling pathway active, even in absence of insulin.

Also, the PST-WT and the variant can bind to the insulin binding pocket at the same time, as their binding locations do not overlap in the insulin binding pocket (FIG. 10B, helix 2 vs. helix 3). The majority of the protein-protein complexes are stabilized through parallel helix-helix stacking at the inter-protein interface, as found here in the docked complexes of PST-NΔ3 (the peptide with SEQ ID NO. 1) and the insulin receptor.

Further embodiments provide compositions with the peptide comprising SEQ ID NO. 1 which can be formulated as a drug to improve glucose tolerance in insulin resistant and obese individuals or in diabetic patients without weight reduction.

Further embodiments provide methods in which the peptide comprising SEQ ID NO. 1 is used for administering to a patient in need for treatment of insulin resistance.

Further embodiments provide methods for controlling patient's sensitivity to insulin by controlling the levels of PST expression as PST blocks insulin from regulating the carbohydrates uptake by cells. As feeding a high fat diet to a mammal leads to obesity, hyperinsulinemia and inflammation, blocking PST may lead to maintaining insulin sensitivity even in obese mammals.

Thus, a method is provided for preventing a damage caused by dietary fat. This method comprises blocking PST in the mammal. The method can be accomplished with a composition comprising the peptide with SEQ ID NO. 1, a peptide which is a functional equivalent for the peptide with SEQ ID NO. 1 in its binding to the insulin receptor and by other peptides and antibodies which can antagonize the binding of the PST with SEQ ID NO. 2 to the insulin receptor and/or some other functions of the PST with SEQ ID NO. 2.

Obesity can be dissociated from insulin resistance as long as inflammation is suppressed if the PST function in a mammal is blocked. The presence of supraphysiological levels of PST can reconnect obesity with insulin resistance by inducing inflammation. In the absence of PST, patients can be insulin sensitive despite obesity.

Further methods are provided for controlling inflammation in a patient. In these methods, the inflammation is reduced or prevented by treating the patient with a composition comprising a compound which block PST with SEQ ID NO. 2. In some embodiments, the method is performed with the peptide comprising, consisting essentially of, or consisting of SEQ ID NO. 1. Further embodiments are performed with a variant of the peptide with SEQ ID NO. 1 which is a functional equivalent of the peptide with SEQ ID NO. 1 and has retained at least 50% binding activity to the insulin receptor of the binding activity of the peptide with SEQ ID NO. 1 to the insulin receptor.

Methods are provided for improving glucose tolerance and insulin sensitivity. In these methods, a patient is treated with a composition comprising a peptide containing SEQ ID NO. 1 either alone or in combination with other treatments known for ameliorating insulin resistance and controlling glucose metabolism.

The invention will be now explained in more detail by the way of the following non-limiting examples.

### Example 1—Methods

Laboratory Animals.

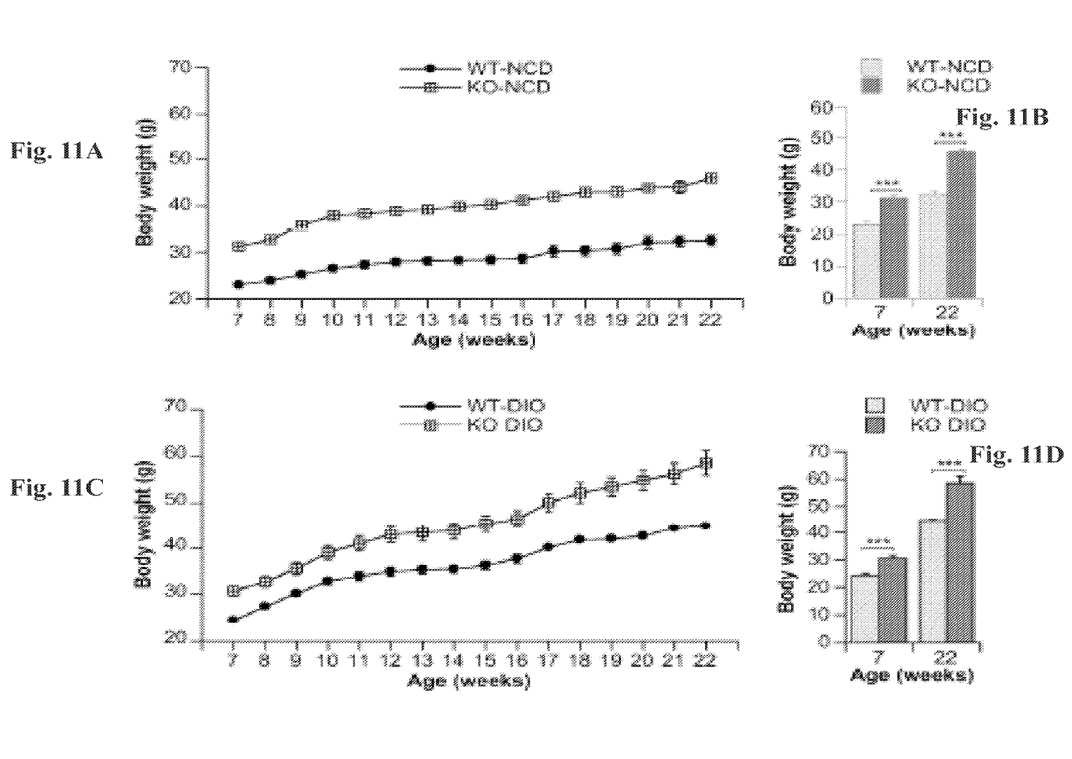

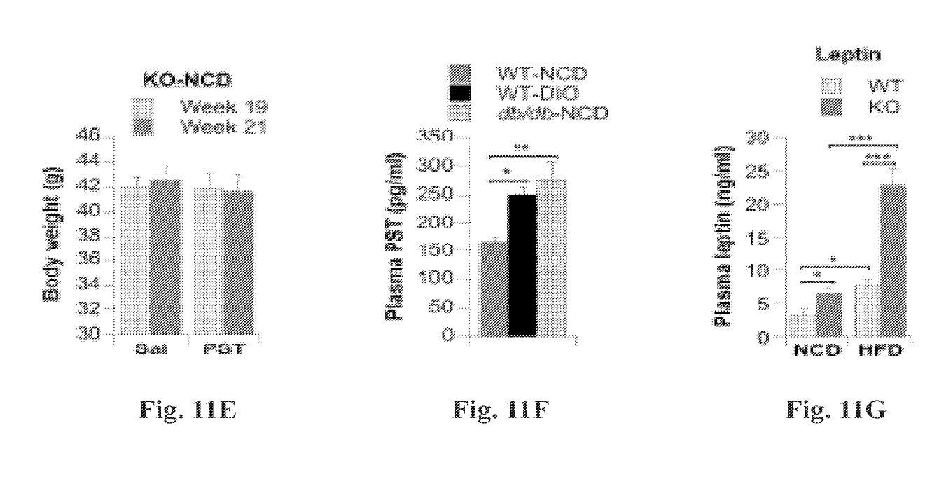

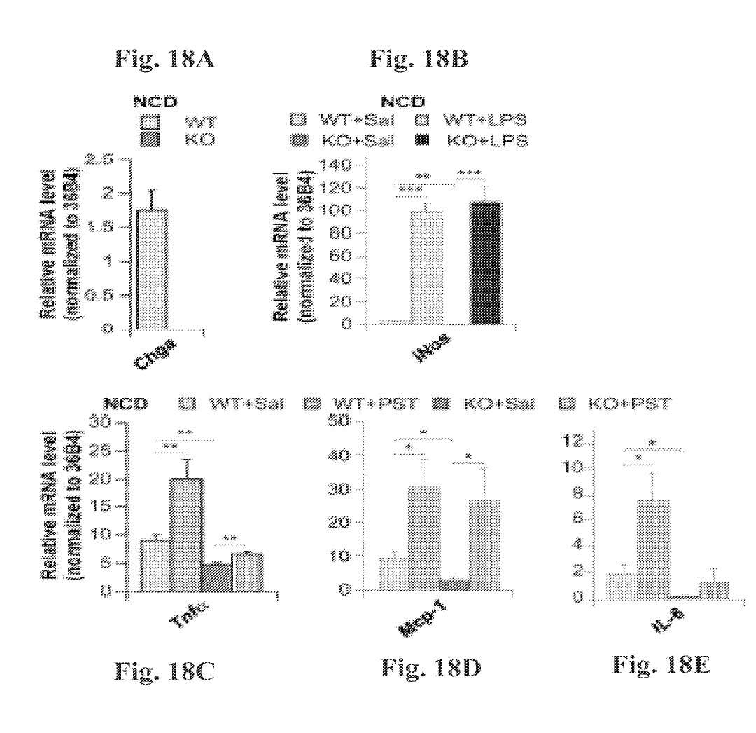

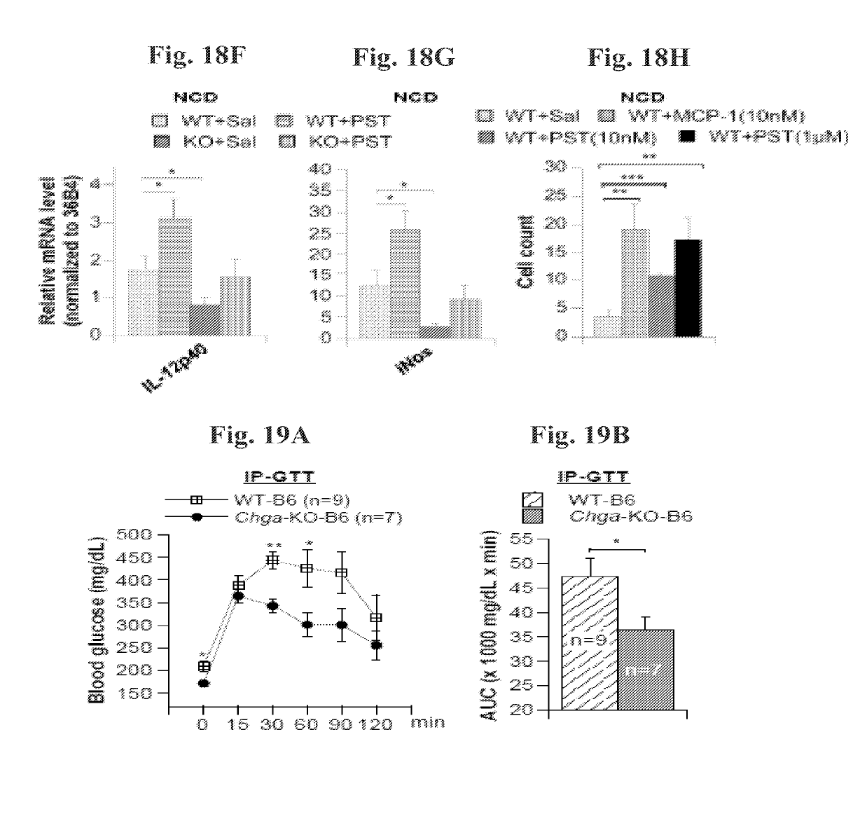

Male WT (control) and Chga-KO mice on a stable mixed genetic background (50% 129svJ; 50% C57BL/6J), for >50 generations were used. Only in experiments shown in FIGS. 11F and 18H, mice were on a C57BL/6J background. Animals were kept in a 12 hours dark/light cycle. The Institutional Animal Care and Utilization Committee approved all the procedures.

Diets.

Mice were fed ad libitum for 12-16 weeks with a high fat diet (HFD: 60% of calories from fat; Research Diets, Inc. D12492). Some mice were also fed a normal chow diet (NCD: 14% calorie from fat; LabDiet 5P00).

Synthetic Peptides.

Wild-type human PST (CHGA₂₇₃₋₃₀₁: PEGKGEQEHSQQKEEEEEMAVVPQGLFRG-amide, SEQ ID NO. 2) and PST variant PSTv1 (also known as PSTNΔ3; CHGA₂₇₆₋₃₀₁: KGEQEHSQQKEEEEEMAVVPQGLFRG-amide, SEQ ID NO. 1) were synthesized by the solid phase method and purified by reverse phase HPLC to at least 95% homogeneity (GenScript Corporation, NJ).

GTT, ITT and Clamp Studies.

For glucose tolerance tests (GTT), glucose (1 mg/g) was injected intraperitoneally (IP-GTT) (at time zero) or gavaged orally (O-GTT) after a 12-hours fast. Tail-vein glucose levels were measured at −30, 0, 15, 30, 60, 90, and 120 minutes. For insulin tolerance tests (ITT), insulin (0.4 mU/g) was injected intraperitoneally and blood glucose levels were measured at the indicated time points. Chronic PST (5 μg/g/day) and PSTv1 (10 μg/day) treatments were carried out for 15 days. For acute treatment, either peptides or saline were injected 30 minutes before glucose or insulin injection. Synergy Software Kaleidagraph Version 4.0 calculated the area under the curve (AUC) for each line curve.

The hyperinsulinemic-euglycemic clamp protocol was adapted. Insulin-stimulated glucose disposal rate (IS-GDR) was calculated as: IS-GDR=glucose disposal rate (GDR; during the clamp)−basal GDR. This value represents a measurement of the increase in GDR from the basal value due to insulin infusion. Since basal hepatic glucose production [HGP]=basal GDR, it was calculated that the total GDR during the clamp as glucose infusion rate (GIR)+HGP (during the clamp). During the clamps, insulin was infused at a constant rate of 12 mU/kg/min. The suppression of HGP by insulin was calculated.

Immunoblotting.

Tissues were homogenized in a buffer containing phosphatase and protease inhibitors. Homogenates were subjected to SDS-PAGE and immunoblotted. Primary antibodies for phosphorylated and total Akt, AMPK, FoxO1, and JNK were from Cell Signaling Technology (Beverly, Mass.). Actin and Srebp-1 antibodies were from Santa Cruz Biotechnology (CA).

Preparation and Culture of Intraperitoneal Macrophages.

Peritoneal macrophages were isolated after thioglycollate (3% solution in water) stimulation as described. Isolated macrophages were cultured in DMEM with 10% FBS for 48 hours with daily medium changes. Cells were then serum-starved overnight in DMEM with low FBS (0.5%) and then exposed to saline, PST (100 nM), or LPS (100 ng/ml) for 4 hours. At the end of incubation, the cultures were subjected to RNA extraction.

In Vitro Chemotaxis Assay.

In vitro chemotaxis assays were performed as previously described. Briefly, thioglycollate-activated peritoneal macrophages were isolated from 8 week-old male C57BL/6J mice and pretreated with the indicated concentrations of PST for 2 hours at 37° C. in serum-free RPMI. For migration analysis, 200,000 peritoneal macrophages were placed in each of the upper chamber of a 4-μm polycarbonate filter (24-transwell format; Corning, Tewksbury, Mass.), whereas compounds resuspended in serum-free RPMI at the indicated concentrations were placed in the lower chamber. After 3 hours of migration at 37° C., cells were fixed in 4% paraformaldehyde, stained with DAPI and counted under the microscope.

Real Time PCR.

RNA from tissues was extracted using a kit (RNeasy Plus, Qiagen, Valencia, Calif.). After DNase digestion, 200 ng of RNA was transcribed into cDNA in a 20-μl reaction using a High Capacity cDNA Archive kit and this cDNA was amplified. PCR (25-μl reactions) contained 5 μl of cDNA, 2×SYBR Green PCR Master Mix, and 400 nM of each primer. Differences in the threshold cycle number (ΔCt) between the target gene, the housekeeping gene Gapdh and the ribosomal protein gene 36B4 were used to calculate differences in expression. Primer sequences are provided below.

| (SEQ ID NO. 3) | |
|---|---|
| Acc F: | TAA TGG GCT GCT TCT GTG ACT C |
| (SEQ ID NO. 4) | |
| Acc R: | CTC AAT ATC GCC ATC AGT CTT G |
| (SEQ ID NO. 5) | |
| Acox F: | GTC GAC CTT GTT CGC CA |
| (SEQ ID NO. 6) | |
| Acox R: | GGT TCC TCA GCA CGG CTT |
| (SEQ ID NO. 7) | |
| Arg-1 F: | ATG GAA GAG ACC TTC AGC TAC |
| (SEQ ID NO. 8) | |
| Arg-1 R: | GCT GTC TTC CCA AGA GTT GGG |
| (SEQ ID NO. 9) | |
| 36b4 F: | AGA TGC AGC AGA TCC GCA T |
| (SEQ ID NO. 10) | |
| 36b4R: | GTT CTT GCC CAT CAG CAC C |
| (SEQ ID NO. 11) | |
| Cd11c F: | ACA CAG TGT GCT CCA GTA TGA |
| (SEQ ID NO. 12) | |
| Cd11c R: | GCC CAG GGA TAT GTT CAC AGC |
| (SEQ ID NO. 13) | |
| Chga F: | AGG AGC GTC TGT CCA GAG AA |
| (SEQ ID NO. 14) | |
| Chga R: | CAG GCT CTC TAG CTC CTG GT |
| (SEQ ID NO. 15) | |
| Cpt1 F: | CAG GAT TTT GCT GTC AAC CTC |
| (SEQ ID NO. 16) | |
| Cpt1 R: | GAG CAT CTC CAT GGC GTA G |
| (SEQ ID NO. 17) | |
| Gapdh F: | TAT GTC GTGGAG TCT ACT GGT GT |
| (SEQ ID NO. 18) | |
| Gapdh R: | GTC ATC ATA CTT GGC AGG TTT CT |
| (SEQ ID NO. 19) | |
| G6Pase F: | GTT GAA CCA GTC TCC GAC CA |
| (SEQ ID NO. 20) | |
| G6Pase R: | CGA CTC GCT ATC TCC AAG TGA |
| (SEQ ID NO. 21) | |
| IL-1b F: | AAA TAC CTG TGG CCT TGG GC |
| (SEQ ID NO. 22) | |
| IL-1b R: | CTT GGG ATC CAC ACT CTC CAG |
| (SEQ ID NO. 23) | |
| IL-6 F: | CCA GAG ATA CAA AGA AAT GAT GG |
| (SEQ ID NO. 24) | |
| IL-6 R: | ACT CCA GAA GAC CAG AGG AAA T |
| (SEQ ID NO. 25) | |
| IL-10 F: | TGA ATT CCC TGG GTG AGA AG |
| (SEQ ID NO. 26) | |
| IL-10 R: | TCA CTC TTC ACC TGC TCC ACT |
| (SEQ ID NO. 27) | |
| IL-12p40 F: | CCA GAG ACA TGG AGT CAT AG |
| (SEQ ID NO. 28) | |
| IL-12p40 R: | AGA TGT GAG TGG CTC AGA GT |
| (SEQ ID NO. 29) | |
| iNos F: | GAG GCC CAG GAG GAG AGA GAT CCG |
| (SEQ ID NO. 30) | |
| iNos R: | TCC ATG CAG ACA ACC TTG GTG TTG |
| (SEQ ID NO. 31) | |
| Mcp-1 F: | AGG TCC CTG TCA TGC TTC TG |
| (SEQ ID NO. 32) | |
| Mcp-1 R: | GCT GCT GGT GAT CCT CTT GT |
| (SEQ ID NO. 33) | |
| Mg1-1 F: | ATG ATG TCT GCC AGA GAA CC |
| (SEQ ID NO. 34) | |
| Mg1-1 R: | ATC ACA GAT TTC AGC AAC CTT A |
| (SEQ ID NO. 35) | |
| Mg1-2 F: | CAG AAC TTG GAG CGG GAA GAG |
| (SEQ ID NO. 36) | |
| Mg1-2 R: | TTC TTG TCA CCA TTT CTC ATC TCC T |
| (SEQ ID NO. 37) | |
| Pepck F: | CTG CAT AAC GGT CTG GAC TTC |
| (SEQ ID NO. 38) | |
| Pepck R: | CAG CAA CTG CCC GTA CTC C |
| (SEQ ID NO. 39) | |
| Srebp-1c F: | GGA GCC ATG GAT TGC ACA TT |
| (SEQ ID NO. 40) | |
| Srebp-1c R: | CTT CCA GAG AGG AGG CCA G |
| (SEQ ID NO. 41) | |
| Tnfα F: | CCA GAC CCT CAC ACT CAG ATC |
| (SEQ ID NO. 42) | |
| Tnfα R: | CAC TTG GTG GTT TGC TAC GAC |
| (SEQ ID NO. 43) | |
| Ym1 F: | GGG CAT ACC TTT ATC CTG AG |
| (SEQ ID NO. 44) | |
| Ym1 R: | CCA CTG AAG TCA TCC ATG TC |

Determination of Plasma Cytokines.

Plasma cytokine concentrations were determined by a multiplex system from Quansys Biosciences (Logan, Utah).

Statistics.

Data are expressed as mean±SEM. Statistical analyses were performed using Student's t tests, as well as one- and two-way ANOVA followed by Dunnett's post hoc test when appropriate. Statistical significance was concluded at p<0.05. Statistics were computed with the program InStat (GraphPad Software, Inc. San Diego, Calif.).

### Example 2—Effects of PST Deficiency and Supplementation on Obesity

The deficiency of Chga proprotein causes obesity in Chga-KO mice. As shown in FIG. 11A, body weights of WT and Chga-KO mice (with mixed genetic background) on NCD were taken every week starting from week 7 until week 22 (2-way ANOVA: Strain, p<0.0001; Age, p<0.0001; Interaction, p<0.004; n=9), and as shown in FIG. 11B, the initial weight at week 7 and final weight at week 22 were compared. As shown in FIG. 11C, body weights of WT and Chga-KO mice on HFD were taken every week starting from week 7 till week 22 (2-way ANOVA: Strain, p<0.0001; Age, p<0.0001; Interaction, p=0.16; n=13), and as shown in FIG. 11D, the initial weight at week 7 and final weight at week 22 were compared. As shown in FIG. 11E, the effects of PST administration to WT and Chga-KO mice on NCD for 2 weeks (from week 19 to week 21) on body weight gain were shown. As shown in FIG. 11F, plasma PST levels were measured in 6 month old WT-NCD mice, or WT-DIO or in obese-diabetic db/db mice (all mice with C57/BL6 background) (n=6). As shown in FIG. 11G, four month old WT and Chga-KO mice with mixed genetic background were fed NCD or HFD for 12 weeks. Plasma leptin levels were measured by ELISA (n=7). “*” Indicates comparison between the indicated groups. “*”: P<0.05, “**”: p<0.01, “***”: p<0.001.

At 7 weeks of age on normal chow diet (NCD), Chga-KO mice were heavier than WT mice (31.28 g vs. 23.08 g). From week 7 to 22, there was an additional increase in body weight gain compared to WT mice (from 23.08 g to 32.53 g for WT mice versus 31.28 g to 45.96 g for Chga-KO mice) during the same period of growth (FIG. 11A-11B). A similar trend holds true for mice on a high fat diet (HFD). After 15 weeks of HFD feeding, Chga-KO-DIO (KO-DIO) mice gained more weight than WT-DIO mice (from 24.4 g to 44.84 g for WT-DIO versus from 30.72 g to 58.59 g for Chga-KO mice) (FIG. 11C-11D). PST deficient KO-DIO mice also ate more on a HFD (2.94 vs. 2.11 gm/day, p<0.0001) than WT-DIO mice. PST administration to Chga-KO mice on NCD for 2 weeks did not change body weight (FIG. 11E).

With the increase in obesity, plasma levels of PST were elevated in WT-DIO mice, similar to the levels seen in obese and diabetic db/db mice (FIG. 11F). Since obesity in Chga-KO mice occurred in the absence of PST expression and PST supplementation did not change body weight, PST is not involved in the development of obesity. Plasma leptin level increased in both WT-DIO and KO-DIO mice compared to NCD-fed mice (FIG. 11G) but the level in KO-DIO was the highest due to the (i) increase in adipose mass (compared to WT-DIO mice) and (ii) absence of PST-mediated negative regulation of leptin production.

### Example 3—Obese Chga-KO Mice Display Improved Glucose Tolerance and Insulin Sensitivity, Dependent Upon the Absence of PST

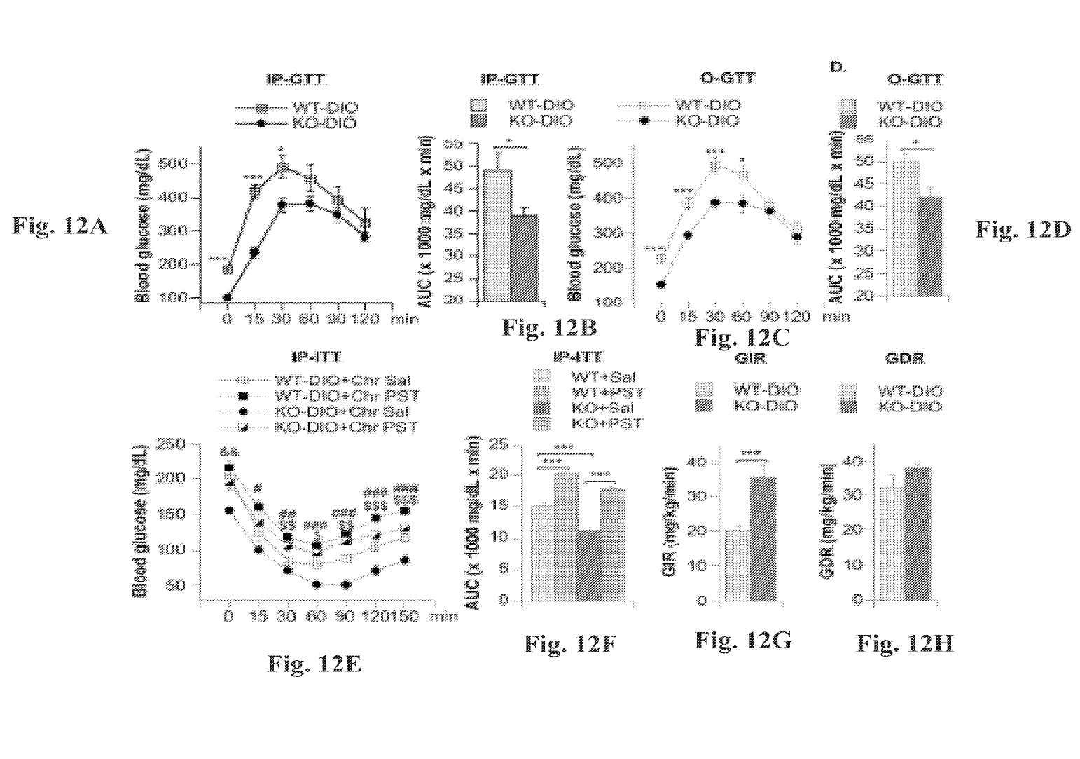

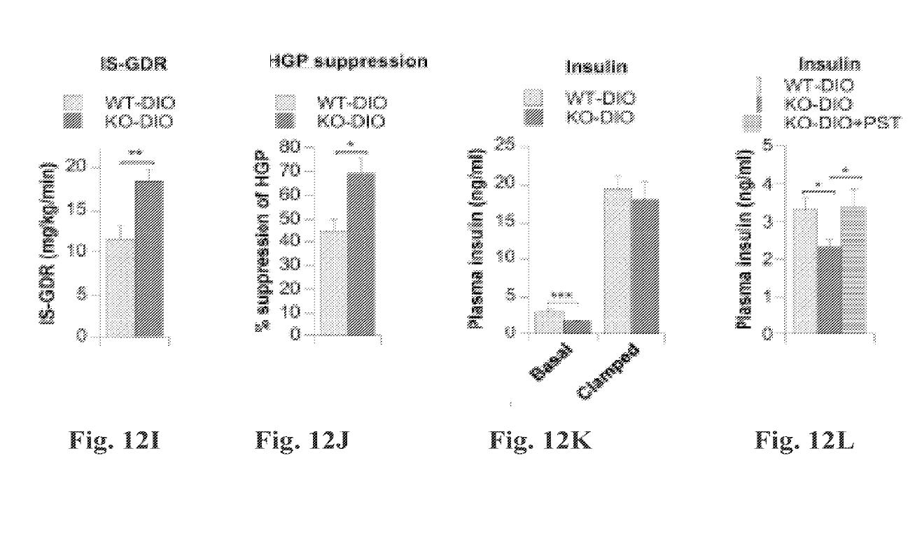

Despite increased obesity, KO-DIO mice maintained insulin sensitivity as shown by GTT and ITT. FIGS. 12A-12L show that Chga-KO mice on HFD display improved glucose tolerance and insulin sensitivity, dependent upon the absence of PST. WT-DIO and KO-DIO male mice with mixed genetic background were fasted for 12 hrs and subjected to IP-GTT (2-way ANOVA: Strain, p<0.0001; Time, p<0.0001; Interaction, p<0.045; n=8, as shown in FIGS. 12A and 12B), oral GTT (2-way ANOVA: Strain, p<0.0001; Time, p<0.0001; Interaction, p=0.09; n=8, as shown in FIGS. 12C and 12D), or IP-ITT (2-way ANOVA: Treatment (WT), p<0.0001; Time, p<0.0001; Interaction, p=0.56; Treatment (KO), p<0.0001; Time, p<0.0001; Interaction, 0.43; n=9, as shown in FIGS. 12E and 12F), and AUC for glucose excursions were determined (see FIGS. 12B, 12D and 12F). As shown in FIG. 12E, WT-DIO and KO-DIO mice were treated with intraperitoneal PST (5 μg/g BW) for 15 days, fasted for 12 hrs after the last day of injection and subjected to GTT. GTT and the corresponding AUC were shown in FIGS. 12E and 12F, respectively. Body weight-matched WT-DIO and KO-DIO mice were fasted for 12 hrs and subjected to clamp studies to determine glucose infusion rate (GIR, shown in FIG. 12G), glucose disposal rate (GDR, shown in FIG. 12H), (I) insulin-stimulated GDR (IS-GDR, shown in FIG. 12I) and % suppression of hepatic glucose production (HGP) (n=8-9, shown in FIG. 12J). Fasting (12 hrs) basal levels of plasma insulin before (FIG. 12K) and after (FIG. 12L) chronic PST treatment (5 μg/g BW/day, IP for 15 days) (n=6) were shown. “&” Indicates comparison between WT-DIO+Sal and KO-DIO+Sal, “$” indicates comparison between WT-DIO+Sal and WT-DIO+PST, “#” indicates comparison between KO-DIO+Sal and KO-DIO+PST, and “*” indicates comparison between the indicated groups. “*, $ or #”: P<0.05, “**, &&, ## or $$”: p<0.01, “***, ### or &&&”: p<0.001.

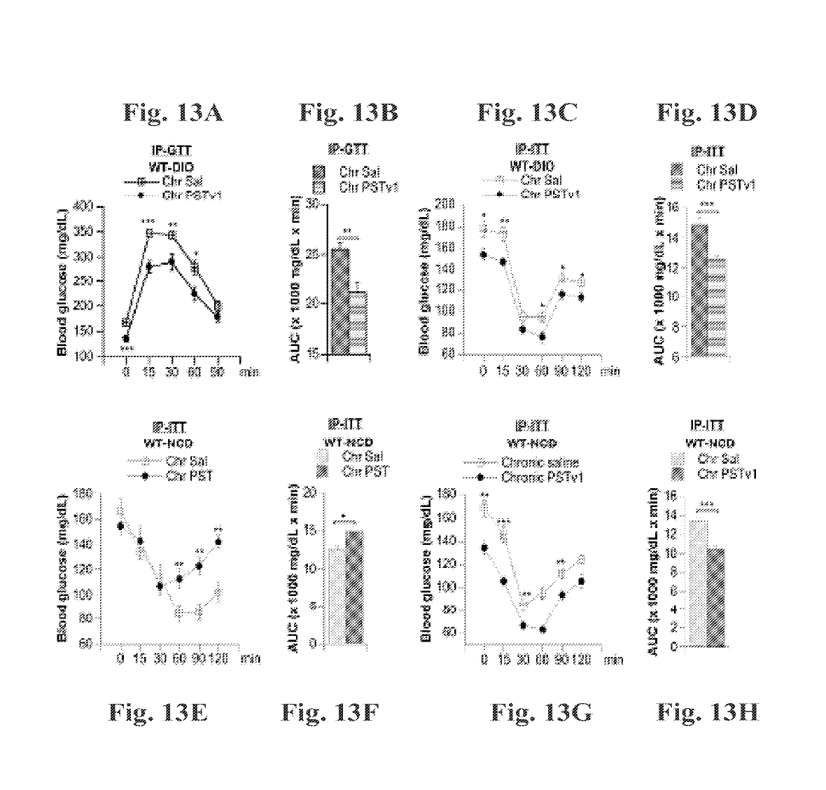

Both IP-GTT (FIGS. 12A and 12B) and O-GTT (FIGS. 12C and 12D) demonstrated improved glucose tolerance, whereas IP-ITT (FIGS. 12E and 12F) showed increased insulin sensitivity in KO-DIO mice. Decreased AUC (area under the curve) for corresponding GTT (FIGS. 12B and 12D) and ITT (FIG. 12F) established that KO-DIO mice were more glucose tolerant and insulin sensitive than WT-DIO mice. Of note, fasting glucose levels (at time zero) in KO-DIO mice were consistently lower than WT-DIO mice (FIGS. 12A, 12C, 12E and FIGS. 13A and 13C). It appears that the lack of PST in KO-DIO mice helps to maintain their insulin sensitivity on HFD, as this phenotype was abolished upon chronic supplementation of PST (FIGS. 12E and 12F). Similarly, when PST was supplemented to WT or Chga-KO mice on a NCD, PST caused insulin resistance (FIGS. 13E and 13F).

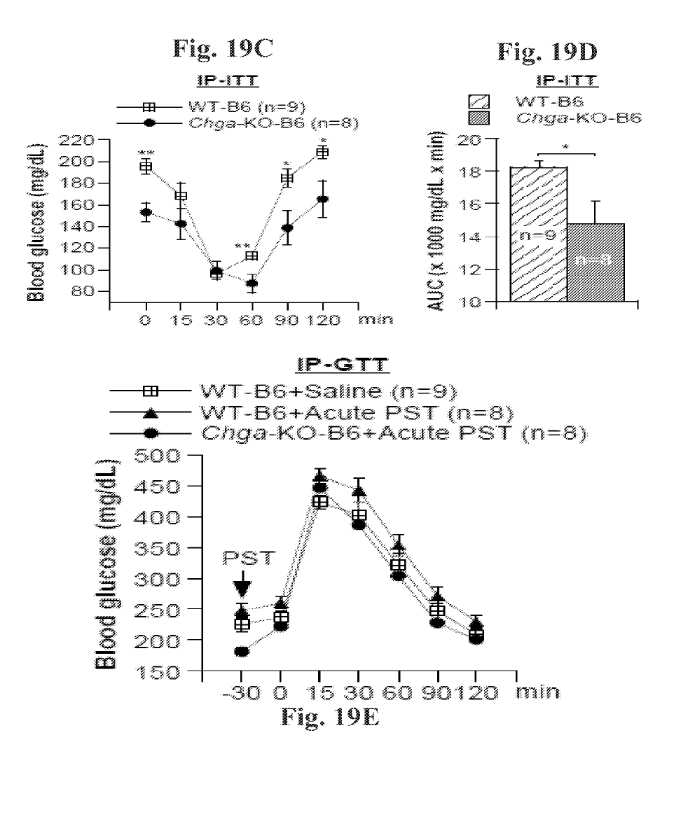

These data emphasize the point that PST treatment causes insulin resistance without causing obesity, and that obesity occurring in the absence of PST does not induce insulin resistance. These results in mice with a mixed genetic background were also corroborated in mice with a C57/BL6J background (FIGS. 19A-19E).

The GTT and ITT findings of insulin sensitivity in KO-DIO mice were reinforced by hyperinsulinemic-euglycemic clamp studies, where KO-DIO mice displayed an increased GIR and suppressed HGP by insulin. The basal GDR was similar between WT-DIO and KO-DIO mice but IS-GDR was higher in KO-DIO compared to WT-DIO mice (FIG. 12G-12J). Fasting plasma insulin concentrations in KO-DIO mice were lower than that of WT-DIO mice (FIG. 12K). Since supplementation of PST raised fasting glucose (FIGS. 12E and 12F) and insulin levels in KO-DIO mice over WT-DIO levels (FIG. 12L), our results support the notion that the deficiency of PST in Chga-KO mice prevented the development of hyperglycemia and hyperinsulinemia in KO-DIO mice.

### Example 4—The Effects of PST, Much Like Anti-Insulin, were Reversed by a PST-Variant Peptide

To combat anti-insulin like effects of PST, PSTv1 was created by deleting three N-terminal residues of native PST. PSTv1 treatment of WT-DIO mice (10 μg/g/day) for 15 days lowered fasting plasma glucose levels (FIGS. 13A and 13C) as well as improved glucose tolerance (FIGS. 13A and 13B) and insulin sensitivity (FIGS. 13C and 13D) possibly by competing against endogenous PST.

Treatment with the PST variant PSTv1 improves, whereas PST worsens, glucose tolerance and insulin sensitivity in WT-DIO and WT-NCD mice. WT-DIO mice were treated with saline or PSTv1 (10 μg/g BW/day) for 15 days. Weight-matched DIO mice were fasted for 12 hrs and subjected to IP-GTT (2-way ANOVA: Treatment, p<0.0001; Time, p<0.0001; Interaction, p=0.17, n=6, as shown in FIG. 13A) and IP-ITT (2-way ANOVA: Treatment, p<0.0001; Time, p<0.0001; Interaction, p=0.62 n=9, as shown in FIG. 13C) and AUCs for glucose excursions were determined (as shown in FIGS. 13B and 13D). NCD fed WT mice WT-NCD) were treated with saline or PST (5 μg/g BW) for 15 days, fasted for 12 hrs and subjected to IP-ITT (2-way ANOVA: Treatment, p<0.0001; Time, p<0.0001; Interaction, p<0.0004; n=8, as shown in 13E). The corresponding AUC was shown in FIG. 13F. WT-NCD mice were also treated with saline or PSTv1 (10 μg/g BW) for 15 days, fasted for 12 hrs and subjected to IP-ITT (2-way ANOVA: Treatment, p<0.0001; Time, p<0.0001; Interaction, p<0.02 n=8, as shown in 13G). The corresponding AUC was shown in 13H. “*” Indicates comparison between the indicated groups. “*”: P<0.05, “**”: p<0.01, “***”: p<0.001.

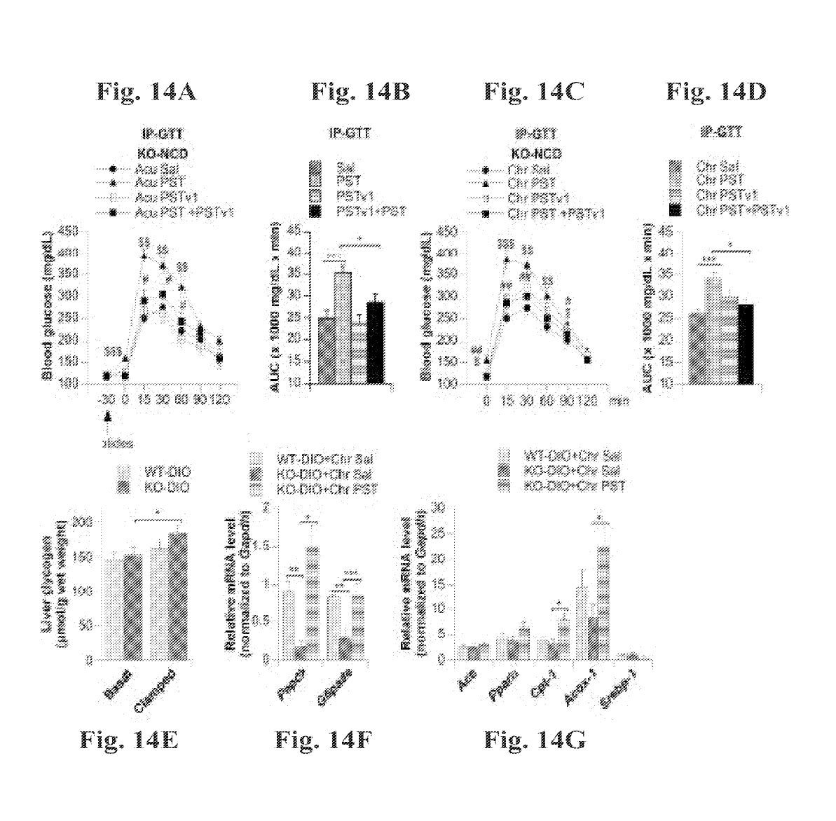

While chronic PST supplementation to both WT and Chga-KO mice on a NCD induced insulin resistance (FIGS. 13E and 13F and FIGS. 14A-14D), chronic PSTv1 improved insulin sensitivity (FIGS. 13G and 13H). In NCD-fed Chga-KO mice (KO-NCD), the hyperglycemic effect of PST was attenuated by concomitant injection of PSTv1 (FIGS. 14A and 14B). Moreover, chronic supplementation of PSTv1 reversed the effects of chronic PST treatment (FIGS. 14C and 14D). Of note, PSTv1 alone had no effect on Chga-KO mice, presumably because of the lack of endogenous PST with which PSTv1 could compete (FIGS. 14A-14D). These results demonstrate PST deficiency as the primary reason for insulin sensitivity and protection against diet-induced insulin resistance in Chga-KO mice.

To determine acute effects, NCD fed Chga-KO mice (KO-NCD) were fasted for 12 hrs, treated with saline, PST (5 μg/g BW), PSTv1 (10 μg/g BW) or PST+PSTv1 for 30 min (−30 min) before injecting glucose (0 min) for (FIG. 14A) IP-GTT (2-way ANOVA: Treatment (Sal vs PST), p<0.0001, Time, p<0.0001; Interaction, p<0.002; Treatment (PST vs PSTv1), p<0.0001; Time, p<0.0001; Interaction, p<0.005; n=7). The corresponding AUC is shown in (FIG. 14B).

To determine chronic effects, KO-NCD mice were treated with saline, PST (5 μg/g BW), PSTv1 (10 μg/g BW) or PST+PSTv1 for 15 days, fasted for 12 hrs after the last of injection and subjected to (FIG. 14C) GTT (2-way ANOVA: Treatment (Sal vs PST), Time, p<0.0001; Interaction, p<0.05; Treatment (PST vs PSTv1), p<0.0001; Time, p<0.0001; Interaction, p<0.01; n=7). The corresponding AUC is shown in (FIG. 14D). FIG. 14E reports liver glycogen at basal and during clamp in DIO mice (n=6). FIG. 14F reports chronic effects of PST (5 μg/g BW for 15 days) on expression of hepatic gluconeogenic genes (Pepck and G6pase) in DIO mice (n=6). FIG. 14G reports chronic effects of PST (5 μg/g BW for 15 days) on expression of hepatic lipid metabolic (Acc, Ppara, Cpt-1, Acox-1 and Srebp-1) genes in DIO mice (n=6). “$” Denotes comparison between saline and PST, “#” denotes comparison between PSTv1 and PST+PSTv1, and “*” denotes comparison between the indicated groups. “*, $ or #”: P<0.05, “**, $$ or ##”: p<0.01, “*** or $$$”: p<0.001.

### Example 5—Effects of PST Deficiency and HFD on Metabolic Regulation and Insulin Signaling

KO-DIO mice displayed increased hepatic glycogen storage and reduced gluconeogenic gene expression. Under the hyperinsulinemic condition during clamps, KO-DIO mice assimilated more glycogen in the liver than WT-DIO mice (FIG. 14E), which helped remove circulating glucose. The mRNA levels of gluconeogenic genes (Pepck and G6pase) in the liver were significantly lower in KO-DIO mice (FIG. 14F), and were reversed by treatment with PST (FIG. 14F). In addition, analysis of lipid metabolism gene expression revealed no difference in the mRNA levels of genes for lipogenesis (Acc and Srebp-1) and lipid oxidation (Ppara, Cpt-1 and Acox) between WT-DIO and KO-DIO mice (FIG. 14G), whereas PST supplementation increased the expression of Acox-1 and Cpt-1.

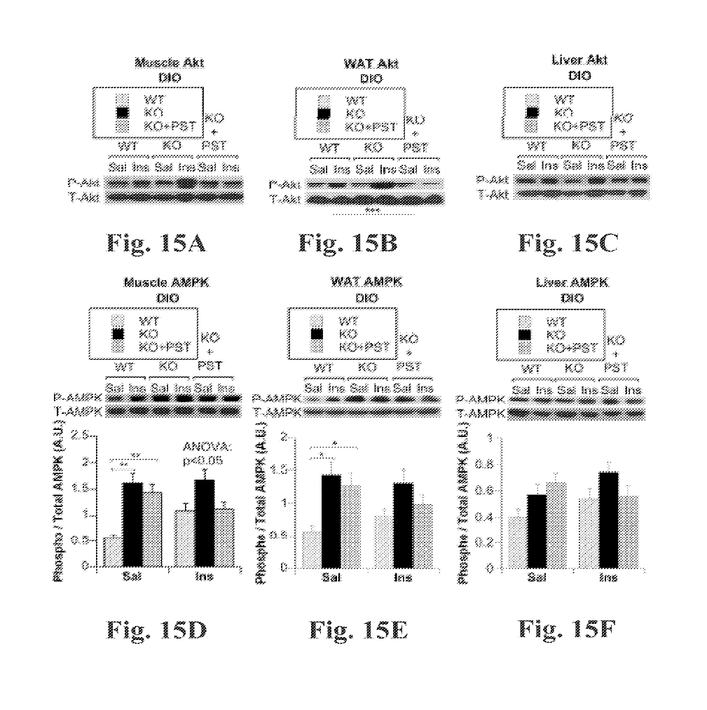

Among the metabolic signals that were significantly improved in Chga-KO mice were Akt and Srebp1c, which are likely to be involved in producing the insulin sensitive phenotype of these animals. Phosphorylation and activation of Akt, an important component of the insulin signaling pathway was analyzed by Western blot. Akt phosphorylation in muscle (FIG. 15A) and white adipose tissue (WAT; FIG. 15B) was not significantly increased by insulin in WT-DIO mice (due to insulin resistance), but was robustly stimulated by insulin in KO-DIO mice (FIGS. 15A and 15B). Weight-matched saline-treated WT-DIO or KO-DIO and PST-treated KO-DIO mice were fasted for 12 hrs, injected with insulin (0.4 mU/g BW, IP) for 10 min and sacrificed for tissue collection. Tissues were homogenized, lysates were subjected to SDS-PAGE and immunoblotted for p-Akt in muscle (14A, n=4), WAT (14B, n=4) and liver (14C, n=4) and for p-AMPK in muscle (14D), WAT (14E) and liver (14F). “*” Denotes comparison between the indicated groups. “*”: P<0.05, “**”: p<0.01, “***”: p<0.001.

In the liver, insulin significantly enhanced Akt phosphorylation in KO-DIO mice when compared with the saline-treated controls (FIG. 15C). The fold response to insulin was reduced in WT-DIO livers (because of insulin resistance) but was higher in KO-DIO livers compared to WT-DIO livers (FIG. 15C). In contrast, PST supplementation to KO-DIO mice completely inhibited stimulation of phosphorylation by insulin (FIGS. 15A-15C).

In addition to Akt, the activity of other signaling pathway components, including AMPK, FoxO1, and Jnk was analyzed. AMPK phosphorylation was improved in the muscle and WAT of KO-DIO mice (but not in the liver) (FIGS. 15D-15F), which might have contributed to an increase in glucose disposal and decrease in inflammation in KO-DIO mice. However, PST supplementation to KO-DIO mice did not change the overall pattern of AMPK signaling. Weight-matched saline-treated WT-DIO or KO-DIO and PST-treated KO-DIO mice were fasted for 12 hrs, injected with insulin (0.4 mU/g BW, IP) for 10 min and sacrificed for tissue collection. Tissues were homogenized, lysates were subjected to SDS-PAGE and immunoblotted for p-Akt in muscle (15A, n=4), WAT (15B, n=4) and liver (15C, n=4) and for p-AMPK in muscle (15D), WAT (15E) and liver (15F). “*” Denotes comparison between the indicated groups. “*”: P<0.05, “**”: p<0.01, “***”: p<0.001.

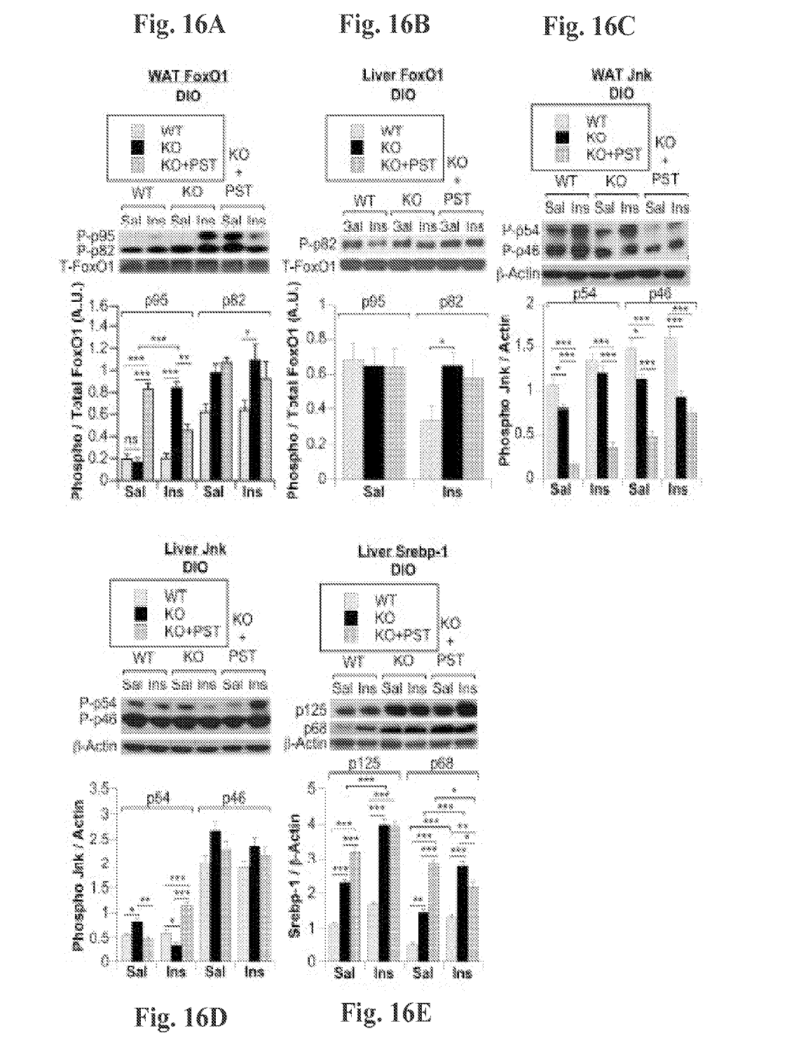

The insulin-stimulated phosphorylation of FoxO1, downstream of Akt (82 kDa pFoxO1 and 95 kDa pFoxO1), in KO-DIO mice was higher in WAT and the liver than corresponding insulin-treated WT-DIO tissues (FIGS. 16A and 16B). Weight-matched saline-treated WT-DIO or KO-DIO and PST-treated KO-DIO mice were fasted for 12 hrs, injected with insulin (0.4 mU/g BW, IP) for 10 min and sacrificed for tissue collection. Tissues were homogenized, lysates were subjected to SDS-PAGE and subsequently immunoblotted to detect phospho-p95-FoxO1 and phospho-p82-FoxO1 signals in WAT (16A, n=4) and phospho-p82-FoxO1 signals in liver (16B, n=4), phospho-p54-Jnk and phospho-p46-Jnk signals in WAT (16C, n=4) and liver (16D, n=4), and p125-Srebp-1 and p-68-Srebp-1 (mature) signals in liver (16E, n=4). “*” Denotes comparison between the indicated groups. “*”: P<0.05, “**”: p<0.01, “***”: p<0.001.

The PST supplementation modulated FoxO1 signaling in WAT but not in the liver (FIGS. 16A and 16B). In addition, the basal phosphorylation of p46 and p54 Jnk was lower in KO-DIO WAT compared to WT-DIO, suggesting persistent activation of inflammatory signals in WT-DIO mice (FIGS. 16C and 16D). Interestingly, insulin seems to stimulate these negative Jnk signals in WAT for self-regulation. However, PST treatment did not alter hepatic AMPK, FoxO1 or Jnk signaling, suggesting additional PST-independent regulatory mechanisms.

Consistent with reduced gluconeogenesis, the expression level of mature Srebp-1c p68 protein in the liver of insulin-treated KO-DIO mice was higher than the WT-DIO controls (FIG. 16E), suggesting that the processing of the precursor p125 to mature p68 was facilitated by insulin in KO-DIO livers. Compared to WT-DIO mice, basal levels of p125 and p68 were also elevated in the liver (FIG. 16E) of KO-DIO mice. PST treatment reversed the expression pattern of p68 in KO-DIO livers by raising basal levels and decreasing insulin sensitivity (FIG. 16E). As a result, PST treatment decreased the p68 to p125 ratio compared to untreated KO-DIO livers. In other words, PST treatment reduced insulin-stimulated processing of the Srebp-1c precursor p125 to mature p68 in KO-DIO livers.

### Example 6—PST Modulates Adipose Tissue Inflammation

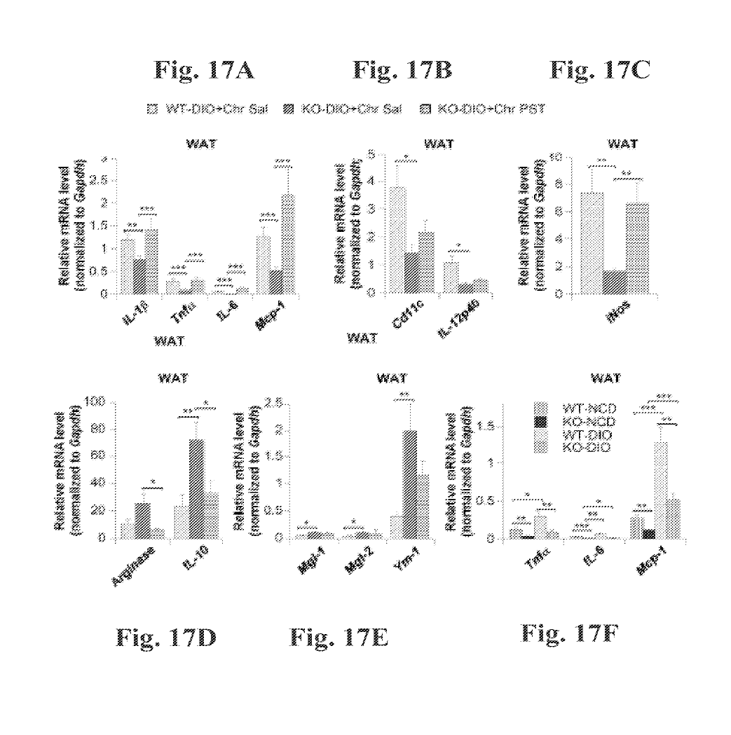

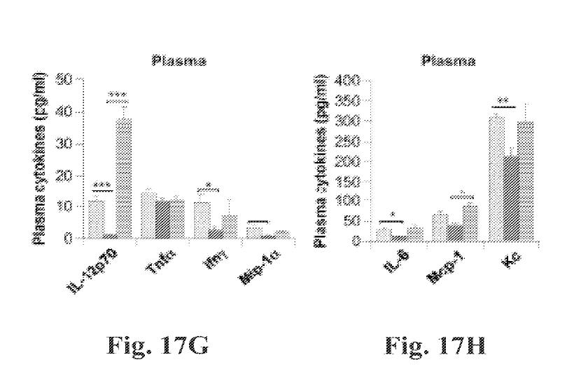

As shown in FIGS. 17A-17H, PST modulates adipose tissue inflammation. A group of KO-DIO mice were injected with saline or PST (5 μg/g BW) for 15 days. Weight-matched saline-treated WT-DIO, KO-DIO and PST-treated KO-DIO mice were fasted for 12 hr and sacrificed. Blood was collected to measure plasma cytokine levels. Tissues were subjected to RNA extraction, cDNA preparation and RT-qPCR analysis for cytokines (FIG. 17A, n=6), pro-inflammatory genes (FIG. 17B: Cd11c & IL-12p40, n=6; FIG. 17C: iNos, n=6), and anti-inflammatory genes (FIG. 17D: Arg-1 & IL-10, n=6; FIG. 17E: Mgl-1, Mgl-2 and Ym1, n=6). Plasma cytokine levels were shown in FIGS. 17G and 17H (n=5). “*” Denotes comparison between the indicated groups. “*”: P<0.05, “**”: p<0.01, “***”: p<0.001.

mRNA levels of the pro-inflammatory cytokines IL-1β, Tnfα, IL-6, and Mcp-1 were reduced in adipose tissue of KO-DIO mice compared to WT-DIO mice (FIG. 17A). Consistent with this trend, the expression of the pro-inflammatory genes Cd11c and IL-12p40 (FIG. 17B) and iNos (FIG. 17C) were also lower in KO-DIO adipose tissue than WT-DIO mice. Accordingly, PST treatment caused increased expression of the pro-inflammatory genes IL-1β Tnfα, IL-6, Mcp-1 and iNos (FIGS. 17A-17C). Conversely, expression of anti-inflammatory genes such as Arg-1, IL-10, Mgl-1, Mgl-2, and Ym1 in adipose tissue was higher in KO-DIO mice than WT control animals (FIGS. 17D and 17E). PST treatment significantly reduced the expression of Arg-1 and IL-10, and displayed a trend toward downregulating Ym-1, but had no effect on Mgl-1 or Mgl-2 (FIGS. 17D and 17E). The adipose tissue expression of Tnfα, IL-6 and Mcp-1 genes was reduced in KO mice in either NCD or DIO conditions compared to WT, although DIO increased levels in both backgrounds (FIG. 17F). The subdued state of inflammation found in KO mice contributes to their improved insulin sensitivity.

Corresponding to reduced inflammatory gene expression, several cytokines were detected at lower levels in the plasma of KO-DIO compared to WT-DIO mice. Levels of IL-12p70, Ifnγ, Mip-1, IL-6 and Kc showed significant decreases, whereas Mcp-1 levels showed a trend to be lower, and Tnfα levels were unchanged (FIGS. 17G and 17H). PST treatment of KO-DIO mice raised plasma levels of IL-12p70 and Mcp-1 but had no effect on Ifnγ, IL-6, Mip-1α, Kc and Tnfα (FIGS. 17G and 17H).

Overall, expression of most pro-inflammatory markers were reduced whereas anti-inflammatory markers were elevated in KO-DIO mice compared to WT-DIO mice, likely due to deficiency of PST and other Chga-derived peptides.

### Example 7—PST Promotes Macrophage Inflammation and Chemotaxis

As shown in FIGS. 18A-18H, PST promotes macrophage inflammation and chemotaxis. Peritoneal macrophages were isolated from WT-NCD and KO-NCD after thioglycollate injection. After 4-hr exposure to saline, LPS (100 ng/ml) or PST (100 nM), RNAs were extracted and cDNAs were prepared for for qPCR analyses of Chga (FIG. 18A, n=8), iNos (FIG. 18B, n=8), Tnfα (FIG. 18C, n=8), Mcp-1 (FIG. 18D, n=8), IL-6 (FIG. 18E, n=8), IL-12p40 (FIG. 18F, n=8) and iNos genes (FIG. 18G, n=8). Effects of LPS stimulation on expression of iNos are shown in (18B) and the effects of PST are shown in FIGS. 18C-18G. (FIG. 18H) Effects of PST on chemotaxis of activated macrophages from NCD-fed C57BL/6J mice (n=5). “*” Denotes comparison between the indicated groups. “*”: P<0.05, “**”: p<0.01, “***”: p<0.001.

Elevated numbers of adipose tissue macrophages are associated with obesity, increased inflammation and reduced insulin sensitivity. Chga mRNA expression was identified in peritoneal macrophages of WT mice (FIG. 18A). LPS (lipopolysaccharide) caused robust expression of the pro-inflammatory gene iNos (˜100-fold) in both WT and Chga-KO macrophages (FIG. 18B). This was a reference point for comparing PST effects on expression of other inflammatory markers. The basal expression of iNos, Tnfα, Mcp-1, IL-6, and IL-12p40 and iNos was lower in Chga-KO macrophages, and PST treatment caused a smaller increase in expression than in WT macrophages (FIGS. 18B-18F). Specifically, PST increased the expression of Tnfα and Mcp-1, but had no effect on the expression of IL-6, IL-12p40 and iNos genes in Chga-KO macrophages (FIGS. 18C-18G). Developmental adjustments in Chga-KO mice may have lowered the potential for induction of these genes by PST. It is also possible that supplementation of other Chga-derived peptides may be necessary to produce the full complement of expression in Chga-KO mice.

Consistent with induction of inflammatory gene expression, PST induced chemotaxis of macrophages in vitro. Using peritoneal macrophage cultures, we compared the chemotactic ability of the chemokine Mcp-1 versus PST. PST treatment led to significantly higher levels of chemotaxis compared to saline-treated controls, although with lower potency than Mcp-1 (chemotaxis induced by 10 nM PST was 44% that of 10 nM Mcp-1) (FIG. 18H).

As shown in FIGS. 19A-19E, further tests were conducted with wild type and Chga-KO mice on the C57BL/6 genetic background. FIGS. 19A-19E report GTT and ITT in mice with C57BL/6 genetic background. Three month old WT and Chga-KO mice were fed HFD for 12 weeks. Body weight-matched mice were fasted for 12 hrs and subjected to IP-GTT (FIG. 19A) and ITT (FIG. 19C) and the corresponding AUCs for glucose excursions were determined (FIGS. 19B and 19D).

As shown in FIG. 19E, four month old Chga-KO mice were fed HFD for 12 months,

fasted for 12 hrs, injected with PST (5 μg/g BW, IP) at −30 min and subjected to GTT after injecting glucose at 0 min. *: P<0.05, **: p<0.01, ***: p<0.001.

In summary, these results reinforce the notion that (i) PST alone negatively regulates insulin sensitivity in several tissues, and as a result, PST supplementation can produce insulin resistance, and (ii) along with another Chga-produced peptide, PST upregulates proinflammatory signals that are lacking in Chga-KO mice.

### Example 8—Blocking PST Helps in Managing Diabetes and Inflammation

PST deficiency renders NCD-fed Chga-KO mice more insulin sensitive than WT mice. A GTP-binding protein-coupled-receptor mediated signaling pathway leading to activation of conventional DAG/Ca²⁺-dependent PKC and downregulation of mature Srebp-1c (p68), is thought to play a role in the anti-insulin effects of PST. PST could also modulate ER-stress by interacting with BiP/GRP78. Feeding a HFD creates obesity, leading to hyperinsulinemia and inflammation. PST-deficient KO-DIO mice are more obese than WT-DIO mice but remain more insulin sensitive as assessed by GTT, ITT and clamp studies (FIG. 12). Insulin sensitivity was compromised by PST replacement, suggesting that the absence of PST in Chga-KO mice not only enhanced insulin action but also prevented further damage by dietary fat.

Obesity can be dissociated from insulin resistance as long as inflammation is suppressed. The presence of supraphysiological levels of PST can reconnect obesity with insulin resistance by inducing inflammation. In the absence of PST, animals could be insulin sensitive despite obesity. This is reminiscent of rosiglitazone-treated WT-DIO mice, which are insulin sensitive but obese.

The HFD feeding does not suppress the insulin-sensitive phenotype of Chga-KO mice, the PST-deficiency in Chga-KO mice provides protection against HFD-induced insulin resistance, and PST is an endogenous pro-inflammatory factor that affects insulin action independent of obesity. Obesity caused by dietary fat cannot induce inflammation and insulin resistance in the absence of PST and another Chga-produced peptide.

It appears that the PST deficiency provides benefits to obese mice by (i) enhancing hepatic glycogen storage and decreasing glucose production through stimulation of Akt signaling and suppression of gluconeogenic genes via increased expression of Srebp-1c proteins, and (ii) increasing glucose disposal by muscle via increased AMPK and insulin-stimulated Akt signaling (FIGS. 12 and 15), and by (iii) suppressing macrophage-mediated inflammation in adipose tissue (FIGS. 17 and 18). Moreover, the PST deficiency may decrease ER stress.

The PST deficiency also increased insulin-stimulated pFoxO1-p95 signals in WAT of KO-DIO mice, which was reversed after PST supplementation (FIG. 16A). Since increased phosphorylation of FoxO1 (inactive form) in WAT favors adipogenesis through PPARγ, these results are consistent with the greater adiposity of Chga-KO mice.

The increased Akt and AMPK activities in WAT in KO-DIO mice (FIG. 15) could potentially provide the benefit of dampening the inflammation induced by a HFD. This is because both Akt and AMPK can suppress the inflammatory responses of resident macrophages in adipose tissue and modulate cytokine production by adipocytes, macrophages and neutrophils. These results corroborate this notion and suggest that supraphysiological levels of PST can impose an inflammatory burden and thereby disrupt glucose homeostasis. The profiling of pro- and anti-inflammatory markers and cytokines suggests that the adipose tissue of KO-DIO mice was less inflamed than WT-DIO mice. Since PST treatment of peritoneal macrophage cultures (obtained from WT and Chga-KO mice on NCD) increased the expression of Tnfα, Mcp-1, IL-6, IL-12p40 and iNos (FIGS. 18C-18G), this suggests a direct effect of PST on the behavior of peritoneal macrophages.

The expression of these genes was much lower in Chga-KO macrophages. Treatment of Chga-KO macrophages with PST elevated their expression but not to the extent of PST-treated WT macrophages (FIG. 18C-18G). Moreover, PST provoked macrophage chemotaxis in vitro (FIG. 18H). It is possible that other Chga-derived peptides also contributed to the regulation of inflammatory gene expression, leading to a partial PST-stimulated response in Chga-KO macrophages compared to WT macrophages. However, in terms of insulin resistance, PST alone can fully account for the insulin resistance conferred to KO-DIO mice. It is possible that the diabetogenic effects of a supraphysiological level of PST may result from the combined stimulation of two different pathways; inflammation and ER stress because PST reduces the adaptive unfolded protein response through binding to the ER chaperone, GRP78.

Finally, to implicate a direct in vivo role of PST in the regulation of insulin sensitivity, WT-DIO mice were injected with the PST variant, PSTv1, to block the effect of native PST (which is upregulated in DIO mice). PSTv1 lacks the first three N-terminal residues of native PST and was designed to block PST-mediated inhibition of glucose uptake and leptin secretion in 3T3-L1 adipocytes (manuscript in preparation). Chronic PSTv1 treatment lowered fasting plasma glucose levels in WT-DIO mice and improved glucose tolerance (FIGS. 3A&B) and insulin sensitivity (FIGS. 3C&D). These results suggested that in WT-DIO mice where the level of PST is high, PSTv1 administration could compete with the native PST and thereby phenocopy Chga-KO mice. From this perspective, PSTv1 could serve as an anti-diabetic agent.

The deficiency of PST in Chga-KO mice provides an anti-inflammatory environment leading to prevention of HFD-induced insulin resistance. As a result, insulin sensitivity is maintained even in obese KO-DIO mice. Thus, obesity manifests insulin resistance only in the presence of PST, and in its absence obesity is dissociated from insulin resistance. The chain of events following suppression of PST action that contributes to improved glucose homeostasis can be defined as follows: (i) promotion of insulin signaling and suppression of inflammatory cytokine production through increased PI-3-K/Akt/Foxo-1 signaling and (ii) suppression of hepatic gluconeogenesis by increased expression of mature Srebp-1c. Future studies will be directed to determine whether administration of PSTv1 to diabetic animals will improve insulin sensitivity by reducing PST-induced inflammation and ER stress.

### REFERENCES

1. Tatemoto K, Efendic S, Mutt V, Makk G, Feistner G J, Barchas J D: Pancreastatin, a novel pancreatic peptide that inhibits insulin secretion. Nature 1986; 324:476-478
2. Gayen J R, Saberi M, Schenk S, Biswas N, Vaingankar S M, Cheung W W, Najjar S M, O'Connor D T, Bandyopadhyay G, Mahata S K: A novel pathway of insulin sensitivity in chromogranin a null mice: A crucial role for pancreastatin in glucose homeostasis. J Biol Chem 2009; 284:28498-28509
3. Sanchez-Margalet V, Gonzalez-Yanes C, Najib S, Santos-Alvarez J: Metabolic effects and mechanism of action of the chromogranin A-derived peptide pancreastatin. Regul Pept 2010; 161:8-14
4. O'Connor D T, Cadman P E, Smiley C, Salem R M, Rao F, Smith J, Funk S D, Mahata S K, Mahata M, Wen G, Taupenot L, Gonzalez-Yanes C, Harper K L, Henry R R, Sanchez-Margalet V: Pancreastatin: multiple actions on human intermediary metabolism in vivo, variation in disease, and naturally occurring functional genetic polymorphism. J Clin Endocrinol Metab 2005; 90:5414-5425
5. Borgs M, Bollen M, Keppens S, Yap S H, Stalmans W, Vanstapel F: Modulation of basal hepatic glycogenolysis by nitric oxide. Hepatology 1996; 23:1564-1571
6. Sugita H, Kaneki M, Tokunaga E, Sugita M, Koike C, Yasuhara S, Tompkins R G, Martyn J A: Inducible nitric oxide synthase plays a role in LPS-induced hyperglycemia and insulin resistance. Am J Physiol Endocrinol Metab 2002; 282:E386-394
7. Hutson N J, Brumley F T, Assimacopoulos F D, Harper S C, Exton J H: Studies on the alpha-adrenergic activation of hepatic glucose output. I. Studies on the alpha-adrenergic activation of phosphorylase and gluconeogenesis and inactivation of glycogen synthase in isolated rat liver parenchymal cells. J Biol Chem 1976; 251:5200-5208
8. Biswas N, Friese R S, Gayen J R, Bandyopadhyay G, Mahata S K, O'Connor D T: Discovery of a Novel Target for the Dysglycemic Chromogranin A Fragment Pancreastatin: Interaction with the Chaperone GRP78 to Influence Metabolism. PLoS One 2014; 9:e84132
9. Bandyopadhyay G K, Lu M, Avolio E, Siddiqui J A, Gayen J R, Wollam J, Vu C U, Chi N W, O'Connor D T, Mahata S K: Pancreastatin-dependent inflammatory signaling mediates obesity-induced insulin resistance Diabetes 2014; in press
10. Menting J G, Whittaker J, Margetts M B, Whittaker L J, Kong G K, Smith B J, Watson C J, Zakova L, Kletvikova E, Jiracek J, Chan S J, Steiner D F, Dodson G G, Brzozowski A M, Weiss M A, Ward C W, Lawrence M C: How insulin engages its primary binding site on the insulin receptor. Nature 2013; 493:241-245
11. Chen R, Li L, Weng Z: ZDOCK: an initial-stage protein-docking algorithm. Proteins 2003; 52:80-87
12. Winkler H, Fischer-Colbrie R: The chromogranins A and B: the first 25 years and future perspectives. Neuroscience 1992; 49:497-528
13. Taupenot L, Harper K L, O'Connor D T: Mechanisms of disease: The chromogranin-secretogranin family. New Engl J Med 2003; 348:1134-1149
14. Montero-Hadjadje M, Vaingankar S, Elias S, Tostivint H, Mahata S K, Anouar Y: Chromogranins A and B and secretogranin II: evolutionary and functional aspects. Acta Physiol (Oxf) 2008; 192:309-324
15. Bartolomucci A, Possenti R, Mahata S K, Fischer-Colbrie R, Loh Y P, Salton S R: The extended granin family: structure, function, and biomedical implications. Endocr Rev 2011; 32:755-797
16. Mahata S K, O'Connor D T, Mahata M, Yoo S H, Taupenot L, Wu H, Gill B M, Parmer R J: Novel autocrine feedback control of catecholamine release. A discrete chromogranin A fragment is a noncompetitive nicotinic cholinergic antagonist. J Clin Invest 1997; 100:1623-1633
17. Mahata S K, Mahata M, Wakade A R, O'Connor D T: Primary structure and function of the catecholamine release inhibitory peptide catestatin (chromogranin A344-364): Identification of amino acid residues crucial for activity. Mol Endocrinol 2000; 14:1525-1535
18. Wen G, Mahata S K, Cadman P, Mahata M, Ghosh S, Mahapatra N R, Rao F, Stridsberg M, Smith D W, Mahboubi P, Schork N J, O'Connor D T, Hamilton B A: Both rare and common polymorphisms contribute functional variation at CHGA, a regulator of catecholamine physiology. Am J Hum Genet 2004; 74:197-207
19. Mahata S K, Mahata M, Fung M M, O'Connor D T: Catestatin: a multifunctional peptide from chromogranin A. Regul Pept 2010; 162:33-43
20. Tatemoto K, Efendic S, Mutt V, Makk G, Feistner G J, Barchas J D: Pancreastatin, a novel pancreatic peptide that inhibits insulin secretion. Nature 1986; 324:476-478
21. O'Connor D T, Cadman P E, Smiley C, Salem R M, Rao F, Smith J, Funk S D, Mahata S K, Mahata M, Wen G, Taupenot L, Gonzalez-Yanes C, Harper K L, Henry R R, Sanchez-Margalet V: Pancreastatin: multiple actions on human intermediary metabolism in vivo, variation in disease, and naturally occurring functional genetic polymorphism. J Clin Endocrinol Metab 2005; 90:5414-5425
22. Gayen J R, Saberi M, Schenk S, Biswas N, Vaingankar S M, Cheung W W, Najjar S M, O'Connor D T, Bandyopadhyay G, Mahata S K: A novel pathway of insulin sensitivity in chromogranin a null mice: A crucial role for pancreastatin in glucose homeostasis. J Biol Chem 2009; 284:28498-28509
23. Sanchez-Margalet V, Gonzalez-Yanes C, Najib S, Santos-Alvarez J: Metabolic effects and mechanism of action of the chromogranin A-derived peptide pancreastatin. Regul Pept 2010; 161:8-14
24. Mahapatra N R, O'Connor D T, Vaingankar S M, Sinha Hikim A P, Mahata M, Ray S, Staite E, Wu H, Gu Y, Dalton N, Kennedy B P, Ziegler M G, Ross J, Jr., Mahata S K: Hypertension from targeted ablation of chromogranin A can be rescued by the human ortholog. J Clin Invest 2005; 115:1942-1952
25. Gayen J R, Gu Y, O'Connor D T, Mahata S K: Global disturbances in autonomic function yield cardiovascular instability and hypertension in the chromogranin A null mouse. Endocrinology 2009; 150:5027-5035
26. Dev N B, Gayen J R, O'Connor D T, Mahata S K: Chromogranin A and the autonomic system: Decomposition of heart rate variability by time and frequency domains, along with non-linear characteristics during chromogranin A ablation, with “rescue” by its catestatin. Endocrinology 2010; 151:2760-2768
27. Gayen J R, Zhang K, Ramachandrarao S P, Mahata M, Chen Y, Kim H-S, Naviaux R K, Sharma K, Mahata S K, O'Connor D T: Role of reactive oxygen species in hyperadrenergic hypertension: Biochemical, physiological, and pharmacological evidence from targeted ablation of the chromogranin A gene. Circ Cardiovasc Genet 2010; 3:414-425
28. Bandyopadhyay G K, Vu C U, Gentile S, Lee H, Biswas N, Chi N W, O'Connor D T, Mahata S K: Catestatin (chromogranin A(352-372)) and novel effects on mobilization of fat from adipose tissue through regulation of adrenergic and leptin signaling. J Biol Chem 2012; 287:23141-23151
29. Hotamisligil G S, Shargill N S, Spiegelman B M: Adipose expression of tumor necrosis factor-alpha: direct role in obesity-linked insulin resistance. Science 1993; 259:87-91
30. Yudkin J S, Stehouwer C D, Emeis J J, Coppack S W: C-reactive protein in healthy subjects: associations with obesity, insulin resistance, and endothelial dysfunction: a potential role for cytokines originating from adipose tissue? Arterioscler Thromb Vasc Biol 1999; 19:972-978
31. Festa A, D'Agostino R, Jr., Howard G, Mykkanen L, Tracy R P, Haffner S M: Chronic subclinical inflammation as part of the insulin resistance syndrome: the Insulin Resistance Atherosclerosis Study (IRAS). Circulation 2000; 102:42-47
32. Pradhan A D, Manson J E, Rifai N, Buring J E, Ridker P M: C-reactive protein, interleukin 6, and risk of developing type 2 diabetes mellitus. JAMA 2001; 286:327-334
33. Glass C K, Olefsky J M: Inflammation and lipid signaling in the etiology of insulin resistance. Cell Metab 2012; 15:635-645
34. Pini M, Rhodes D H, Castellanos K J, Cabay R J, Grady E F, Fantuzzi G: Rosiglitazone improves survival and hastens recovery from pancreatic inflammation in obese mice. PloS One 2012; 7:e40944
35. Zhao D, McCully B H, Brooks V L: Rosiglitazone improves insulin sensitivity and baroreflex gain in rats with diet-induced obesity. J Pharmacol Exp Ther 2012; 343:206-213
36. Foryst-Ludwig A, Hartge M, Clemenz M, Sprang C, Hess K, Marx N, Unger T, Kintscher U: PPARgamma activation attenuates T-lymphocyte-dependent inflammation of adipose tissue and development of insulin resistance in obese mice. Cardiovasc Diabetol 2010; 9:64
37. Kim J K: Hyperinsulinemic-euglycemic clamp to assess insulin sensitivity in vivo. Methods Mol Biol 2009; 560:221-238
38. Zhang X, Goncalves R, Mosser D M: The isolation and characterization of murine macrophages. Current protocols in immunology/edited by John E Coligan [et al] 2008; Chapter 14: Unit 14 11
39. Li P, Spann N J, Kaikkonen M U, Lu M, Oh da Y, Fox J N, Bandyopadhyay G, Talukdar S, Xu J, Lagakos W S, Patsouris D, Armando A, Quehenberger O, Dennis E A, Watkins S M, Auwerx J, Glass C K, Olefsky J M: NCoR repression of LXRs restricts macrophage biosynthesis of insulin-sensitizing omega 3 fatty acids. Cell 2013; 155:200-214
40. Gonzalez-Yanes C, Sanchez-Margalet V: Pancreastatin, a chromogranin A-derived peptide, inhibits leptin and enhances UCP-2 expression in isolated rat adipocytes. Cell Mol Life Sci 2003; 60:2749-2756
41. O'Neill L A, Hardie D G: Metabolism of inflammation limited by AMPK and pseudo-starvation. Nature 2013; 493:346-355
42. Bijland S, Mancini S J, Salt I P: Role of AMP-activated protein kinase in adipose tissue metabolism and inflammation. Clin Sci 2013; 124:491-507
43. Galic S, Fullerton M D, Schertzer J D, Sikkema S, Marcinko K, Walkley C R, Izon D, Honeyman J, Chen Z P, van Denderen B J, Kemp B E, Steinberg G R: Hematopoietic AMPK beta1 reduces mouse adipose tissue macrophage inflammation and insulin resistance in obesity. J Clin Invest 2011; 121:4903-4915
44. Sag D, Carling D, Stout R D, Suttles J: Adenosine 5′-monophosphate-activated protein kinase promotes macrophage polarization to an anti-inflammatory functional phenotype. J Immunol 2008; 181:8633-8641
45. Rajaram M V, Ganesan L P, Parsa K V, Butchar J P, Gunn J S, Tridandapani S: Akt/Protein kinase B modulates macrophage inflammatory response to Francisella infection and confers a survival advantage in mice. J Immunol 2006; 177:6317-6324
46. de Oliveira A C, Candelario-Jalil E, Langbein J, Wendeburg L, Bhatia H S, Schlachetzki J C, Biber K, Fiebich B L: Pharmacological inhibition of Akt and downstream pathways modulates the expression of COX-2 and mPGES-1 in activated microglia. J Neuroinflamm 2012; 9:2
47. Hoogendijk A J, Pinhancos S S, van der Poll T, Wieland C W: AMP-activated protein kinase activation by 5-aminoimidazole-4-carbox-amide-1-beta-D-ribofuranoside (AICAR) reduces lipoteichoic acid-induced lung inflammation. J Biol Chem 2013; 288:7047-7052
48. Zhao X, Zmijewski J W, Lorne E, Liu G, Park Y J, Tsuruta Y, Abraham E: Activation of AMPK attenuates neutrophil proinflammatory activity and decreases the severity of acute lung injury. Am J Physiol Lung Cell Mol Physiol 2008; 295:L497-504
49. Huang J B, Ding Y, Huang D S, Liang A J, Zeng W K, Zeng Z P, Qin C Q, Barden B: Inhibition of the PI3K/AKT pathway reduces tumor necrosis factor-alpha production in the cellular response to wear particles in vitro. Artif Organs 2013; 37:298-307
50. Smith M V, Lee M J, Islam A S, Rohrer J L, Goldberg V M, Beidelschies M A, Greenfield E M: Inhibition of the PI3K-Akt signaling pathway reduces tumor necrosis factor-alpha production in response to titanium particles in vitro. J Bone Joint Surg Am 2007; 89:1019-1027
51. Kidd L B, Schabbauer G A, Luyendyk J P, Holscher T D, Tilley R E, Tencati M, Mackman N: Insulin activation of the phosphatidylinositol 3-kinase/protein kinase B (Akt) pathway reduces lipopolysaccharide-induced inflammation in mice. J Pharmacol Exp Ther 2008; 326:348-353
52. Makki K, Froguel P, Wolowczuk I: Adipose Tissue in Obesity-Related Inflammation and Insulin Resistance: Cells, Cytokines, and Chemokines. ISRN inflammation 2013; 2013:139239
53. Xu H, Barnes G T, Yang Q, Tan G, Yang D, Chou C J, Sole J, Nichols A, Ross J S, Tartaglia L A, Chen H: Chronic inflammation in fat plays a crucial role in the development of obesity-related insulin resistance. J Clin Invest 2003; 112:1821-1830
54. Olefsky J M, Glass C K: Macrophages, inflammation, and insulin resistance. Annu Rev Physiol 2010; 72:219-246
55. Bouloumie A, Casteilla L, Lafontan M: Adipose tissue lymphocytes and macrophages in obesity and insulin resistance: makers or markers, and which comes first? Arterioscler Thromb Vasc Biol 2008; 28:1211-1213
56. Biswas N, Friese R S, Gayen J R, Bandyopadhyay G, Mahata S K, O'Connor D T: Discovery of a Novel Target for the Dysglycemic Chromogranin A Fragment Pancreastatin: Interaction with the Chaperone GRP78 to Influence Metabolism. PLoS One 2014; 9:e84132
57. Yamamoto T, Shimano H, Nakagawa Y, Ide T, Yahagi N, Matsuzaka T, Nakakuki M, Takahashi A, Suzuki H, Sone H, Toyoshima H, Sato R, Yamada N: SREBP-1 interacts with hepatocyte nuclear factor-4 alpha and interferes with PGC-1 recruitment to suppress hepatic gluconeogenic genes. J Biol Chem 2004; 279:12027-12035
58. Chakravarty K, Hanson R W: Insulin regulation of phosphoenolpyruvate carboxykinase-c gene transcription: the role of sterol regulatory element-binding protein 1c. Nutr Rev 2007; 65:S47-56
59. Nakae J, Kitamura T, Kitamura Y, Biggs W H, 3rd, Arden K C, Accili D: The forkhead transcription factor Foxo1 regulates adipocyte differentiation. Dev cell 2003; 4:119-129
60. Fan W, Imamura T, Sonoda N, Sears D D, Patsouris D, Kim J J, Olefsky J M: FOXO1 transrepresses peroxisome proliferator-activated receptor gamma transactivation, coordinating an insulin-induced feed-forward response in adipocytes. J Biol Chem 2009; 284:12188-12197

What is claimed is:

1. A composition comprising a peptide comprising the amino acid sequence KGEQEHSQQKEEEEEMAVVPQGLFRG-AMIDE (SEQ ID NO. 1) and at least one excipient.

2. The composition of claim 1, wherein the composition comprises a peptide which consists of the amino acid sequence
(SEQ ID NO. 1)
KGEQEHSQQKEEEEEMAVVPQGLFRG-AMIDE.

3. The composition of claim 1, wherein the composition further comprises at least one of the following: an anti-inflammatory drug, metformin, thiazolidinedione or insulin.

4. The composition of claim 1, wherein the peptide comprises the amino acid sequence in which at least one of the amino acids from KGEQEHSQQKEEEEEMAVVPQGLFRG-AMIDE (SEQ ID NO. 1) is substituted or deleted.

5. The composition of claim 4, wherein the peptide retains at least 50% binding affinity to the insulin receptor of the binding activity for the peptide with SEQ ID NO. 1 to the insulin receptor.

6. The composition of claim 1 in which at least one L-amino acid in the peptide with SEQ ID NO. 1 is substituted with a D-amino acid.

7. The composition of claim 1, formulated for oral administration, intravenous injection or intramuscular injection into a patient.

8. The use of the composition of claim 1, for treating a patient from at least one of the following diseases: insulin resistance, diabetes, type 2 diabetes, inflammation, obesity, non-alcoholic fatty liver disease, atherosclerosis and a cardiovascular disease.

9. The use of the composition of claim 1 for treating a patient selected from the group of patients whose fasting insulin level is 9.0 mlU/ml or higher for treating the patient from at least one of following diseases: insulin resistance, diabetes, type 2 diabetes, inflammation, obesity, non-alcoholic fatty liver disease, atherosclerosis and a cardiovascular disease.

10. The use of the composition of claim 1 for treating a patient from at least one of the following diseases: insulin resistance, diabetes, type 2 diabetes, inflammation, obesity, non-alcoholic fatty liver disease, atherosclerosis and a cardiovascular disease, wherein the composition is administered to the patient in the amount ranging from 5 mg/day to 1 g/day.


SEQUENCE LISTING (44 sequences)

SEQ ID NO: 1 - protein, 26 aa, Artificial Sequence
  Peptide synthetized in the laboratory
KGEQEHSQQKEEEEEMAVVPQGLFRG

SEQ ID NO: 2 - protein, 29 aa, Artificial Sequence
  Pancreastatin
PEGKGEQEHSQQKEEEEEMAVVPQGLFRG

SEQ ID NO: 3 - DNA, 22 bp, Artificial Sequence
  Oligonucleotide synthetized in the laboratory
taatgggctgcttctgtgactc

SEQ ID NO: 4 - DNA, 22 bp, Artificial Sequence
  Oligonucleotide synthetized in the laboratory
ctcaatatcgccatcagtcttg

SEQ ID NO: 5 - DNA, 17 bp, Artificial Sequence
  Oligonucleotide synthetized in the laboratory
gtcgaccttgttcgcca

SEQ ID NO: 6 - DNA, 18 bp, Artificial Sequence
  Oligonucleotide synthesized in the laboratory
ggttcctcagcacggctt

SEQ ID NO: 7 - DNA, 21 bp, Artificial Sequence
  Oligonucleotide synthesized in the laboratory.
atggaagagaccttcagctac

SEQ ID NO: 8 - DNA, 21 bp, Artificial Sequence
  Oligonucleotide synthesized in the library.
gctgtcttcccaagagttggg

SEQ ID NO: 9 - DNA, 19 bp, Artificial Sequence
  Oligonucelotide synthesized in the laboratory.
agatgcagcagatccgcat

SEQ ID NO: 10 - DNA, 19 bp, Artificial Sequence
  Oligonucelotide synthesized in the laboratory.
gttcttgcccatcagcacc

SEQ ID NO: 11 - DNA, 21 bp, Artificial Sequence
  Oligonucleotide synthesized in the laboratory.
acacagtgtgctccagtatga

SEQ ID NO: 12 - DNA, 21 bp, Artificial Sequence
  Oligonucleotide synthesized in the laboratory.
gcccagggatatgttcacagc

SEQ ID NO: 13 - DNA, 20 bp, Artificial Sequence
  Oligonucleotide synthesized in the laboratory.
aggagcgtctgtccagagaa

SEQ ID NO: 14 - DNA, 20 bp, Artificial Sequence
  Oligonucleotide synthesized in the laboratory.
caggctctctagctcctggt

SEQ ID NO: 15 - DNA, 23 bp, Artificial Sequence
  Oligonucleotide synthesized i nthe laboratory.
ctcaggattttgctgtcaacctc

SEQ ID NO: 16 - DNA, 19 bp, Artificial Sequence
  Oligonucelotide synthesized in the laboratory.
gagcatctccatggcgtag

SEQ ID NO: 17 - DNA, 23 bp, Artificial Sequence
  Oligonucleotide synthesized in the laboratory.
tatgtcgtggagtctactggtgt

SEQ ID NO: 18 - DNA, 23 bp, Artificial Sequence
  Oligonucelotide synthesized in the laboratory.
gtcatcatacttggcaggtttct

SEQ ID NO: 19 - DNA, 20 bp, Artificial Sequence
  Oligonucelotide synthesized in the laboratory.
gttgaaccagtctccgacca

SEQ ID NO: 20 - DNA, 21 bp, Artificial Sequence
  Oligonucleotide synthesized in the laboratory.
cgactcgctatctccaagtga

SEQ ID NO: 21 - DNA, 20 bp, Artificial Sequence
  Oligonucleotide synthesized in the laboratory.
aaatacctgtggccttgggc

SEQ ID NO: 22 - DNA, 21 bp, Artificial Sequence
  Oligonucleotide synthesized in the laboratory.
cttgggatccacactctccag

SEQ ID NO: 23 - DNA, 23 bp, Artificial Sequence
  Oligonucleotide synthesized in the laboratory
ccagagatacaaagaaatgatgg

SEQ ID NO: 24 - DNA, 22 bp, Artificial Sequence
  Oligonucleotide synthesized in the laboratory
actccagaagaccagaggaaat

SEQ ID NO: 25 - DNA, 21 bp, Artificial Sequence
  Oligonucleotide synthesized in the laboratory.
tcactcttcacctgctccact

SEQ ID NO: 26 - DNA, 23 bp, Artificial Sequence
  Oligonucleotide synthesized in the laboratory.
ccagagatacaaagaaatgatgg

SEQ ID NO: 27 - DNA, 20 bp, Artificial Sequence
  Oligonucleotide synthesized in the laboratory.
ccagagacatggagtcatag

SEQ ID NO: 28 - DNA, 20 bp, Artificial Sequence
  Oligonucleotide synthesized in the laboratory.
agatgtgagtggctcagagt

SEQ ID NO: 29 - DNA, 24 bp, Artificial Sequence
  Oligonucleotide synthesized in the laboratory.
gaggcccaggaggagagagatccg

SEQ ID NO: 30 - DNA, 24 bp, Artificial Sequence
  Oligonucleotide synthesized in the laboratory.
tccatgcagacaaccttggtgttg

SEQ ID NO: 31 - DNA, 20 bp, Artificial Sequence
  Oligonucleotide sequence synthesized in the laboratory.
aggtccctgtcatgcttctg

SEQ ID NO: 32 - DNA, 20 bp, Artificial Sequence
  Oligonucleotide synthesized in the laboratory.
gctgctggtgatcctcttgt

SEQ ID NO: 33 - DNA, 20 bp, Artificial Sequence
  Oligonucleotide synthesized in the laboratory.
atgatgtctgccagagaacc

SEQ ID NO: 34 - DNA, 22 bp, Artificial Sequence
  Oligonucleotide synthesized in a laboratory.
atcacagatttcagcaacctta

SEQ ID NO: 35 - DNA, 21 bp, Artificial Sequence
  Oligonucleotide synthesized in the laboratory.
cagaacttggagcgggaagag

SEQ ID NO: 36 - DNA, 25 bp, Artificial Sequence
  Oligonucleotide synthesized in the laboratory.
ttcttgtcaccatttctcatctcct

SEQ ID NO: 37 - DNA, 21 bp, Artificial Sequence
  Oligonucleotide synthesized in the laboratory.
ctgcataacggtctggacttc

SEQ ID NO: 38 - DNA, 19 bp, Artificial Sequence
  Oligonucleotide synthesized in the laboratory.
cagcaactgcccgtactcc

SEQ ID NO: 39 - DNA, 20 bp, Artificial Sequence
  Oligonucleotide synthesized in the laboratory.
ggagccatggattgcacatt

SEQ ID NO: 40 - DNA, 19 bp, Artificial Sequence
  Oligonucleotide synthesized in the laboratory.
cttccagagaggaggccag

SEQ ID NO: 41 - DNA, 21 bp, Artificial Sequence
  Oligonucleotide synthesized in the laboratory.
ccagaccctcacactcagatc

SEQ ID NO: 42 - DNA, 21 bp, Artificial Sequence
  Oligonucleotide synthesized in the laboratory.
cacttggtggtttgctacgac

SEQ ID NO: 43 - DNA, 20 bp, Artificial Sequence
  Oligonucleotide synthesized in the laboratory.
gggcatacctttatcctgag

SEQ ID NO: 44 - DNA, 20 bp, Artificial Sequence
  Oligonucleotide synthesized in the laboratory.
ccactgaagtcatccatgtc